SEQUENCE LISTING (69 sequences)

SEQ ID NO: 1 - DNA, 23 bp, Artificial Sequence
  single guide RNA
gtagcagccccgcctccccgagg

SEQ ID NO: 2 - DNA, 23 bp, Artificial Sequence
  single guide RNA
gggcgcagtctgcttgcaggcgg

SEQ ID NO: 3 - DNA, 23 bp, Artificial Sequence
  single guide RNA
tgggctagcctgggtgccagtgg

SEQ ID NO: 4 - DNA, 23 bp, Artificial Sequence
  single guide RNA
tgggtgccagtggctgctagcgg

SEQ ID NO: 5 - DNA, 23 bp, Artificial Sequence
  single guide RNA
gcagccctgagagagcgccgggg

SEQ ID NO: 6 - DNA, 23 bp, Artificial Sequence
  single guide RNA
gcagaatctggctccaggagagg

SEQ ID NO: 7 - DNA, 23 bp, Artificial Sequence
  single guide RNA
gacaaagctctgaggtcctgggg

SEQ ID NO: 8 - DNA, 23 bp, Artificial Sequence
  single guide RNA
gaccaaaaatgtcgagtaagtgg

SEQ ID NO: 9 - DNA, 23 bp, Artificial Sequence
  single guide RNA
agagagcgccggggaaggagagg

SEQ ID NO: 10 - DNA, 23 bp, Artificial Sequence
  single guide RNA
gtgaaatggacaaagctctgagg

SEQ ID NO: 11 - DNA, 23 bp, Artificial Sequence
  single guide RNA
ggctctgacaccatgccgggagg

SEQ ID NO: 12 - DNA, 23 bp, Artificial Sequence
  single guide RNA
aggcggggctgctacctccacgg

SEQ ID NO: 13 - DNA, 23 bp, Artificial Sequence
  single guide RNA
ggcggggctgctacctccacggg

SEQ ID NO: 14 - DNA, 23 bp, Artificial Sequence
  single guide RNA
agactgcgcccctcctgccaggg

SEQ ID NO: 15 - DNA, 23 bp, Artificial Sequence
  single guide RNA
gaggggcgcagtctgcttgcagg

SEQ ID NO: 16 - DNA, 23 bp, Artificial Sequence
  single guide RNA
agcgctccagcggcggctgtcgg

SEQ ID NO: 17 - DNA, 23 bp, Artificial Sequence
  single guide RNA
ggaaagccgacagccgccgctgg

SEQ ID NO: 18 - DNA, 23 bp, Artificial Sequence
  single guide RNA
gctttccaattccgccagctcgg

SEQ ID NO: 19 - DNA, 23 bp, Artificial Sequence
  single guide RNA
gctagcagccactggcacccagg

SEQ ID NO: 20 - DNA, 23 bp, Artificial Sequence
  single guide RNA
tgccagtggctgctagcggcagg

SEQ ID NO: 21 - DNA, 23 bp, Artificial Sequence
  single guide RNA
cgcctgccgctagcagccactgg

SEQ ID NO: 22 - DNA, 23 bp, Artificial Sequence
  single guide RNA
gcaggcgtcccctgagcaacagg

SEQ ID NO: 23 - DNA, 23 bp, Artificial Sequence
  single guide RNA
agggctgcttctttttctctggg

SEQ ID NO: 24 - DNA, 23 bp, Artificial Sequence
  single guide RNA
agcagccctgagagagcgccggg

SEQ ID NO: 25 - DNA, 23 bp, Artificial Sequence
  single guide RNA
gattctgcaggtgcactgggtgg

SEQ ID NO: 26 - DNA, 23 bp, Artificial Sequence
  single guide RNA
gtgcactgggtggggatgatcgg

SEQ ID NO: 27 - DNA, 23 bp, Artificial Sequence
  single guide RNA
gtggggatgatcggcgggctagg

SEQ ID NO: 28 - DNA, 23 bp, Artificial Sequence
  single guide RNA
ggacaaagctctgaggtcctggg

SEQ ID NO: 29 - DNA, 23 bp, Artificial Sequence
  single guide RNA
tggacaaagctctgaggtcctgg

SEQ ID NO: 30 - DNA, 23 bp, Artificial Sequence
  single guide RNA
agcaatgcgttgttcaatgaggg

SEQ ID NO: 31 - DNA, 23 bp, Artificial Sequence
  single guide RNA
ggaatgtccccatagatgaaggg

SEQ ID NO: 32 - DNA, 23 bp, Artificial Sequence
  single guide RNA
aggaatgtccccatagatgaagg

SEQ ID NO: 33 - DNA, 23 bp, Artificial Sequence
  single guide RNA
tggggacattcctcccggcatgg

SEQ ID NO: 34 - DNA, 23 bp, Artificial Sequence
  single guide RNA
cggcatggtgtcagagcccctgg

SEQ ID NO: 35 - DNA, 23 bp, Artificial Sequence
  single guide RNA
gacagccgccgctggagcgctgg

SEQ ID NO: 36 - DNA, 23 bp, Artificial Sequence
  single guide RNA
gagaggcccgcgccctctcctgg

SEQ ID NO: 37 - DNA, 23 bp, Artificial Sequence
  single guide RNA
gccagattctgcaggtgcactgg

SEQ ID NO: 38 - DNA, 23 bp, Artificial Sequence
  single guide RNA
aggggctctgacaccatgccggg

SEQ ID NO: 39 - DNA, 23 bp, Artificial Sequence
  single guide RNA
cggctgaggctgggctagcctgg

SEQ ID NO: 40 - DNA, 23 bp, Artificial Sequence
  single guide RNA
agctcctcacataagaggcctgg

SEQ ID NO: 41 - DNA, 23 bp, Artificial Sequence
  single guide RNA
gtctgcaggaggaaaaagaaagg

SEQ ID NO: 42 - DNA, 23 bp, Artificial Sequence
  single guide RNA
gtaggggtccaagtcctccaggg

SEQ ID NO: 43 - DNA, 23 bp, Artificial Sequence
  single guide RNA
agtaggggtccaagtcctccagg

SEQ ID NO: 44 - DNA, 23 bp, Artificial Sequence
  single guide RNA
gtcagagcccctggaggacttgg

SEQ ID NO: 45 - DNA, 23 bp, Artificial Sequence
  single guide RNA
ggctgaggctgggctagcctggg

SEQ ID NO: 46 - DNA, 23 bp, Artificial Sequence
  single guide RNA
cggcgcagctgccctcggggagg

SEQ ID NO: 47 - DNA, 23 bp, Artificial Sequence
  single guide RNA
cgcagctgccctcggggaggcgg

SEQ ID NO: 48 - DNA, 23 bp, Artificial Sequence
  single guide RNA
cgggcgcgccctggcaggagggg

SEQ ID NO: 49 - DNA, 23 bp, Artificial Sequence
  single guide RNA
ggcggtcgccagcgctccagcgg

SEQ ID NO: 50 - DNA, 23 bp, Artificial Sequence
  single guide RNA
ggtcgccagcgctccagcggcgg

SEQ ID NO: 51 - DNA, 23 bp, Artificial Sequence
  single guide RNA
ggcgcgggcctctccttccccgg

SEQ ID NO: 52 - DNA, 23 bp, Artificial Sequence
  single guide RNA
ggagaaagcatatataaagcagg

SEQ ID NO: 53 - DNA, 23 bp, Artificial Sequence
  single guide RNA
gtgtttaggtacacttttactgg

SEQ ID NO: 54 - DNA, 23 bp, Artificial Sequence
  single guide RNA
gcagctgccctcggggaggcggg

SEQ ID NO: 55 - DNA, 23 bp, Artificial Sequence
  single guide RNA
gaagaggaattaaaatatacagg

SEQ ID NO: 56 - DNA, 10 bp, Artificial Sequence
  Kozak sequence
gccrccatgg

SEQ ID NO: 57 - protein, 5 aa, Artificial Sequence
  linker sequence
DGGGS

SEQ ID NO: 58 - protein, 5 aa, Artificial Sequence
  linker sequence
TGEKP

SEQ ID NO: 59 - protein, 4 aa, Artificial Sequence
  linker sequence
GGRR

SEQ ID NO: 60 - protein, 5 aa, Artificial Sequence
  linker sequence
  REPEAT (1)..(5): May be repeated
GGGGS

SEQ ID NO: 61 - protein, 14 aa, Artificial Sequence
  linker sequence
EGKSSGSGSESKVD

SEQ ID NO: 62 - protein, 18 aa, Artificial Sequence
  linker sequence
KESGSVSSEQLAQFRSLD

SEQ ID NO: 63 - protein, 8 aa, Artificial Sequence
  linker sequence
GGRRGGGS

SEQ ID NO: 64 - protein, 9 aa, Artificial Sequence
  linker sequence
LRQRDGERP

SEQ ID NO: 65 - protein, 12 aa, Artificial Sequence
  linker sequence
LRQKDGGGSERP

SEQ ID NO: 66 - protein, 16 aa, Artificial Sequence
  linker sequence
LRQKDGGGSGGGSERP

SEQ ID NO: 67 - protein, 7 aa, Artificial Sequence
  protease cleavage site
  misc_feature (2)..(3): Xaa can be any naturally occurring amino acid
  misc_feature (5)..(5): Xaa can be any naturally occurring amino acid
  MISC_FEATURE (7)..(7): Xaa may be Gly or Ser
EXXYXQX

SEQ ID NO: 68 - protein, 7 aa, Artificial Sequence
  protease cleavage site
ENLYFQG

SEQ ID NO: 69 - protein, 7 aa, Artificial Sequence
  protease cleavage site
ENLYFQS

(19) United States
(12) Patent Application Publication
(10) Pub. No.: US 2018/0148711 A1
(43) Pub. Date: May 31, 2018

(54) GENOME EDITING VECTORS

(71) Applicant: Coda Biotherapeutics, Inc., South San Francisco, CA (US)

(72) Inventors: Mitchell H. FINER, Stow, MA (US); Kenneth P. GREENBERG, Berkeley, CA (US); Justus B. COHEN, Allison Park, PA (US); Joseph C. GLORIOSO, III, Pittsburg, PA (US)

(21) Appl. No.: 15/575,546

(22) PCT Filed: May 27, 2016
(86) PCT No.: PCT/US2016/034660

§ 371 Date: Nov. 20, 2017

Related U.S. Application Data
(60) Provisional application No. 62/167,613, filed on May 28, 2015.
(60) Provisional application No. 62/254,105, filed on Nov. 11, 2015.

(51) Int. Cl. C12N 15/10; C12N 7/00; C12N 9/22; C12N 15/11; C12N 15/113; C12N 15/86

(52) U.S. Cl. CPC C12N 15/102; C12N 7/00; C12N 9/22; C12N 15/111; C12N 15/113; A61K 48/00; C12N 2710/16642; C12N 2310/20; C12N 2330/51; C12N 2310/141; C12N 2800/80; C12N 15/86

(57) ABSTRACT

The present invention generally provides HSV-based genome editing vectors, cell-based compositions, and methods of using the same.

This application claims priority to and benefit of U.S. Provisional Patent Application No. 62/167,613, filed on May 28, 2015 and of U.S. Provisional Patent Application No. 62/254,105, filed on Nov. 11, 2015. The contents of both these applications are herein incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SWCH_003_01WO_SeqList_ST25.txt, date recorded: May 24, 2016, file size 14 kilobytes).

### FIELD OF THE INVENTION

The present invention generally relates to HSV-based vectors, compositions, and related methods of use for genome editing.

### BACKGROUND OF THE INVENTION

Gene transfer technology has wide-ranging utility in a number of applications relating to biological research and the treatment of disease. Central to this technology is a vector for introducing expression cassettes into target cells such that the cassettes can be expressed in the target cells. Examples of such vectors include naked DNA vectors (such as plasmids), viral vectors (such as adeno-associated viral vectors) (Berns et al., Annals of the New York Academy of Sciences, 772, 95-104 (1995)), adenoviral vectors (Bain et al., Gene Therapy, 1, S68 (1994)), herpesvirus vectors (Fink et al., Ann. Rev. Neurosci., 19, 265-87 (1996)), packaged amplicons (Federoff et al., Proc. Nat. Acad. Sci. USA, 89, 1636-40 (1992)), papilloma virus vectors, picornavirus vectors, polyoma virus vectors, retroviral vectors, SV40 viral vectors, vaccinia virus vectors, and other vectors. Once a given type of vector is selected, its genome must be manipulated for use as a background vector, after which it must be engineered to incorporate exogenous polynucleotides.

HSV vectors have not been widely utilized for these therapies as of yet, primarily due to the difficulty in engineering vectors using standard procedures. The standard method for engineering mutant HSV viruses is to cotransfect host cells with the source virus and a polynucleotide comprising the desired mutation flanked by regions homologous to the target site within the HSV genome. Within the host cell, homologous recombination produces desired mutant HSV viruses less than 5% of the time, with the efficiency generally proportionate to the size of the flanking regions. Aside from inherently low efficiency, recombinant viruses often grow at markedly reduced rates vis-a-vis unmodified parental viruses within host cells, and so are easily overgrown. Thus, where the source vector is already deficient for native loci (particularly essential loci) viral growth can be substantially compromised, and the efficiency of recombination reduced accordingly. Therefore, screening plaques for desired recombinants is a laborious process, and the process becomes incrementally more tedious with multideficient HSV vectors, especially so where the desired mutation is not readily selectable.

Methods for increasing the efficiency of recombination have been attempted, but each presents significant drawbacks for quickly developing novel transfer vectors. Site-specific recombinases such as the cre-lox recombination system (reviewed in Kilby et al. Trends in Genetics, 9, 413-21 (1993)), have been employed to facilitate introduction of exogenous material into viral genomes (see, e.g., Gage et al., J. Virol., 66, 6509-15 (1992)). By these methods, exogenous polynucleotides are introduced into a recombinase recognition site previously engineered into the HSV genome. Recombinants can be selected by assaying for a reporter construct also present within the cassette. Furthermore, source viruses in which the recombinase recognition site is in a locus conferring growth benefits (e.g., the tk locus) do not enjoy the growth advantages over recombinants as seen in traditional methods. Thus, recombinase-mediated production of vectors is a more efficient method for producing HSV vectors than the co-precipitation method, routinely producing desired recombinants roughly 10% of the time, and in some instances site-specific recombination can be significantly more efficient (Rasty et al., Meth. Mol. Genet., 7, 114-30 (1995)). Despite the gain in efficiency, recombinase-mediated production of vectors presents two significant drawbacks. The first is that the method necessarily incorporates the entirety of any plasmid containing the desired insertion sequence, generally a bacterial plasmid for cloning a desired exogenous cassette. Of course, this requirement partially obviates the advantage inherent in the HSV genome's potentially large capacity to accommodate foreign DNA. Secondly, and more significantly, because the recombinase recognition site is retained (in duplicate) within a recombinant vector, subsequent rounds of site-specific recombination greatly disrupt the vectors and can result in randomization of the genome. Thus, site-specific recombination is not a preferred method for generating HSV vectors comprising multiple transgenes.

Engineering HSV vectors to include multiple cassettes has proven difficult. Not only are the aforementioned methods inefficient for developing multigene HSV vectors, but certain properties of the HSV genome have thwarted attempts to create multiple insertions using standard means. For example, during replication, the HSV virus reacts to foreign DNA inserted at or near repeat elements. In some cases, a single expression cassette can be effectively introduced, but when the insertion of a second cassette is attempted, the virus reacts by recombining and shuffling its genome, substantially destabilizing the vector. In other instances, insertion of exogenous DNA disrupts viral function as well. Where multiple cassettes contain homologous regions to each other (i.e., a similar promoter, similar polyadenylation sequences, etc.), intragenomic recombination also can occur to destabilize the vector.

In view of the foregoing, the widespread clinical use of HSV vectors has yet to be realized.

### SUMMARY OF THE INVENTION

The present provides polynucleotides, HSV-based vectors, genetically modified cells and related compositions for improved gene therapy of diseases, disorders, and conditions. Without wishing to be bound by any particular theory, the present inventors have discovered that HSV-based vectors provide several advantages compared to other gene therapy methods, including (1) efficient in vivo delivery; (2) large payload capacity that allows for delivery of a single vector comprising a complete gene therapy solution, including templates for gene correction/gene insertion; and (3) a regulatable genome editing platform with increased efficiency and reduced off-target effects.

In various embodiments, an HSV-based vector comprising a CRISPR-Cas system for the treatment, prevention, or amelioration of chronic pain is provided.

In a particular embodiment, an HSV-based vector comprises a first expression cassette that comprises an RNA polymerase II promoter operably linked to a polynucleotide encoding a CRISPR-Cas endonuclease; and a second expression cassette that comprises one or more RNA polymerase III promoters operably linked to one or more guide RNAs.

In various embodiments, an HSV-based vector comprising an inducibly and transiently regulatable CRISPR-Cas system is contemplated.

In a particular embodiment, an HSV-based vector comprises: a first expression cassette that comprises at least one regulatory element for inducible expression and at least one regulatory element for transient expression and a polynucleotide encoding a CRISPR-Cas endonuclease; a second expression cassette that comprises a polynucleotide encoding one or more guide RNAs; and a third expression cassette that comprises a polynucleotide encoding a switch polypeptide that binds to the at least one element for inducible expression.

In a particular embodiment, an HSV-based vector comprises: a first expression cassette that comprises at least one regulatory element for transient expression and a polynucleotide encoding a CRISPR-Cas endonuclease; a second expression cassette that comprises at least one regulatory element for inducible expression and a polynucleotide encoding one or more guide RNAs; and a third expression cassette that comprises a polynucleotide encoding a switch polypeptide that binds to the at least one element for inducible expression; and optionally, at least one regulatory element for transient expression.

In one embodiment, the HSV-based vector comprise one or more guide RNAs that target one or more guide RNA target sites in the genome.

In a certain embodiment, the at least one regulatory element for transient expression comprises one or more guide RNA target sites.

In another embodiment, the at least one regulatory element for transient expression comprises one or more guide RNA target sites and wherein the polynucleotide encoding the CRISPR-Cas endonuclease is flanked by the one or more guide RNA target sites.

In one embodiment, the at least one regulatory element for transient expression comprises one or more guide RNA target sites and wherein the polynucleotide encoding the switch polypeptide is flanked by one or more guide RNA target sites.

In a further embodiment, the HSV-based vector further comprises a polynucleotide encoding a template for altering at least one site in a genome that is flanked by one or more guide RNA target sites.

In one embodiment, the guide RNA target sites flanking any one of the polynucleotide encoding the CRISPR-Cas endonuclease, the polynucleotide encoding the switch polypeptide, and the polynucleotide encoding the template for altering at least one site in the genome are the same.

In a certain embodiment, the guide RNA target sites flanking the polynucleotide encoding the CRISPR-Cas endonuclease, the polynucleotide encoding the switch polypeptide, and the polynucleotide encoding the template for altering at least one site in the genome are the same.

In an additional embodiment, each of the guide RNA target sites flanking the 5′ end of the polynucleotide encoding the CRISPR-Cas endonuclease, the polynucleotide encoding the switch polypeptide, and the polynucleotide encoding the template for altering at least one site in the genome are the same.

In a particular embodiment, each of the guide RNA target site flanking the 3′ end of the polynucleotide encoding the CRISPR-Cas endonuclease, the polynucleotide encoding the switch polypeptide, and the polynucleotide encoding the template for altering at least one site in the genome are the same.

In another embodiment, the guide RNA target site flanking the 5′ end and the guide RNA target site flanking the 3′ end of any one of the polynucleotides encoding the CRISPR-Cas endonuclease, the switch polypeptide, and the template for altering at least one site in the genome are different.

In one embodiment, the guide RNA target site flanking the 5′ end and the guide RNA target site flanking the 3′ end of the polynucleotides encoding the CRISPR-Cas endonuclease, the switch polypeptide, and the template for altering at least one site in the genome are different.

In a particular embodiment, each of the guide RNAs target sites flanking the 5′ end of the polynucleotides encoding the CRISPR-Cas endonuclease, the switch polypeptide, and the template for altering at least one site in the genome are the same; each of the guide RNAs target sites flanking the 3′ end of the polynucleotides encoding the CRISPR-Cas endonuclease, the switch polypeptide, and the template for altering at least one site in the genome are the same; and wherein the guide RNA target site flanking the 5′ end each polynucleotide is different from the guide RNA target site flanking the 3′ end of each of polynucleotide.

In a particular embodiment, the one or more guide RNA target sites in the HSV-based vector are identical to one or more guide RNA target sites in the genome.

In a certain embodiment, the guide RNA target site flanking the 5′ end of each polynucleotide is identical to a guide RNA target site in the genome; wherein the guide RNA target site flanking the 3′ end of each polynucleotide is identical to a guide RNA target site in the genome; and wherein the guide RNA target site flanking the 5′ end each polynucleotide is different from the guide RNA target site flanking the 3′ end of each of polynucleotide.

In a further embodiment, the second expression cassette comprises one or more guide RNAs that recognize and bind to each of the one or more guide RNAs target sites contemplated herein.

In an additional embodiment, the second expression cassette comprises a single guide RNA that recognizes and binds all of the one or more guide RNA target sites contemplated herein.

In a particular embodiment, the second expression cassette comprises a plurality of guide RNAs, wherein each of the plurality of guide RNAs recognizes and binds to one of the one or more guide RNA target sites contemplated herein.

In one embodiment, the first expression cassette comprises an HSV promoter, optionally wherein the HSV promoter is the LATP2 promoter.

In an additional embodiment, the first expression cassette comprises an HSV promoter and an RNA polymerase II promoter operably linked to the polynucleotide encoding the CRISPR-Cas endonuclease.

In a certain embodiment, the RNA polymerase II promoter is a ubiquitous promoter.

In another embodiment, the ubiquitous promoter is selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In a particular embodiment, the RNA polymerase II promoter is a tissue-specific or lineage-specific promoter.

In a further embodiment, the tissue-specific or lineage-specific promoter is selected from the group consisting of: a B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

In a certain embodiment, the tissue-specific or lineage-specific promoter is selected from the group consisting of: an hSYN1 promoter, a TRPV1 promoter, a Naᵥ1.7 promoter, a Naᵥ1.8 promoter, and a Naᵥ1.9 promoter.

In one embodiment, the tissue-specific or lineage-specific promoter is a promoter specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), or the like

In an additional embodiment, the first expression cassette comprises an HSV promoter and/or at least one regulatory element for inducible expression that is operably linked to the polynucleotide encoding the CRISPR-Cas endonuclease.

In a particular embodiment, at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a peroxisome proliferator-activated receptor-gamma (PPAR-γ) promoter, and a peroxide inducible promoter.

In one embodiment, the first expression cassette comprises a polynucleotide encoding a CRISPR-Cas endonuclease selected from the group consisting of: Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In another embodiment, the first expression cassette comprises a polynucleotide encoding a Cas9 polypeptide.

In one embodiment, the first expression cassette comprises a polynucleotide encoding a Cas9 polypeptide isolated from Staphylococcus aureus, Streptococcus pyrogenes, Streptococcus thermophilis, Treponema denticola, and Neisseria meningitidis.

In a particular embodiment, the Cas9 polypeptide comprises one or more mutations in a HNH or a RuvC-like endonuclease domain or the HNH and the RuvC-like endonuclease domains.

In a particular embodiment, the mutant Cas9 polypeptide is a nickase.

In a particular embodiment, the mutant Cas9 polypeptide sequence is from Streptococcus pyogenes and comprises a mutation in the RuvC domain.

In a further embodiment, the mutation is a D10A mutation.

In a certain embodiment, the mutant Cas9 polypeptide sequence is from Streptococcus pyogenes and comprises a mutation in the HNH domain.

In an additional embodiment, the mutation is a D839A, H840A, or N863A mutation.

In one embodiment, the mutant Cas9 polypeptide sequence is from Streptococcus thermophilis and comprises a mutation in the RuvC-like domain.

In another embodiment, the mutation is a D9A mutation.

In one embodiment, the mutant Cas9 polypeptide sequence is from Streptococcus thermophilis and comprises a mutation in the HNH domain.

In a particular embodiment, the mutation is a D598A, H599A, or N622A mutation.

In an additional embodiment, the mutant Cas9 polypeptide sequence is from Treponema denticola and comprises a mutation in the RuvC-like domain.

In a certain embodiment, the mutation is a D13A mutation.

In a certain embodiment, the mutant Cas9 polypeptide sequence is from Treponema denticola and comprises a mutation in the HNH domain.

In a further embodiment, the mutation is a D878A, H879A, or N902A mutation.

In an additional embodiment, the mutant Cas9 polypeptide sequence is from Neisseria meningitidis and comprises a mutation in the RuvC domain.

In a particular embodiment, the mutation is a D16A mutation.

In another embodiment, the mutant Cas9 polypeptide sequence is from Neisseria meningitidis and comprises a mutation in the HNH domain.

In one embodiment, the mutation is a D587A, H588A, or N611A mutation.

In a particular embodiment, the polynucleotide encoding Cas9 is human codon optimized.

In a particular embodiment, the first expression cassette comprises a polynucleotide encoding a Cpf1 polypeptide.

In a particular embodiment, the polynucleotide encoding Cpf1′ is human codon optimized.

In a certain embodiment, the first expression cassette comprises a polynucleotide encoding a Cpf1 polypeptide isolated from Francisella novicida, Acidaminococcus sp. BV3L6, or Lachnospiraceae bacterium ND2006.

In a further embodiment, the Cpf1 polypeptide comprises one or more mutations in a RuvC-like endonuclease domain.

In a certain embodiment, the mutant Cpf1 polypeptide sequence is from Francisella novicida and comprises a mutation in the RuvC-like domain.

In one embodiment, the mutation is a D917A, E1006A, or D1225A mutation.

In another embodiment, the CRISPR-Cas endonuclease is a Cas9 fusion polypeptide or a Cpf1 fusion polypeptide.

In a certain embodiment, the fusion polypeptide comprises one or more functional domains.

In a further embodiment, the CRISPR-Cas endonuclease is a Cas9 fusion polypeptide or a Cpf1 fusion polypeptide.

In a further embodiment, the fusion polypeptide comprises one or more functional domains.

In a certain embodiment, the one or more functional domains is selected from the group consisting of: a histone methylase or demethylase domains, a histone acetylase or deacetylase domains, a SUMOylation domain, a ubiquitylation or deubiquitylation domain, a DNA methylase or DNA demethylase domain, and a nuclease domain.

In an additional embodiment, the nuclease domain is a FOK I nuclease domain.

In one embodiment, the nuclease domain is a TREX2 nuclease domain.

In an additional embodiment, the switch polypeptide comprises a TREX2 domain or is a polypeptide comprising a self-cleaving viral peptide and TREX2.

In a particular embodiment, the one or more guide RNAs are single strand guide RNAS (sgRNAs).

In one embodiment, the one or more guide RNAs are crRNAs.

In a certain embodiment, the second expression cassette further comprises one or more tracRNAs.

In one embodiment, the second expression cassette further comprises one or more RNA polymerase III promoters operably linked to the one or more guide RNAs.

In a particular embodiment, the second expression cassette further comprises one RNA polymerase III promoter operably linked to the one or more guide RNAs.

In one embodiment, the second expression cassette further comprises one RNA polymerase III promoter operably linked to each of the one or more guide RNAs.

In a particular embodiment, the second expression cassette further comprises one RNA polymerase III promoter operably linked to each of the one or more guide RNAs.

In a further embodiment, the second expression cassette further comprises an RNA polymerase III promoter and at least one regulatory element for inducible expression operably linked to the one or more polynucleotides encoding the one or more guide RNAs.

In an additional embodiment, the RNA polymerase III promoter is selected from the group consisting of: a human U6 snRNA promoter, a mouse U6 snRNA promoter, a human H1 RNA promoter, a mouse H1 RNA promoter, and a human tRNA-val promoter.

In a particular embodiment, the at least one regulatory element for inducible expression is selected from the group consisting of: a tetracycline responsive regulatory element and a peroxide inducible regulatory element.

In one embodiment, the third expression cassette comprises a polynucleotide encoding a switch polypeptide selected from the group consisting of: a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

In another embodiment, the third expression cassette comprises an RNA polymerase II promoter operably linked to the polynucleotide encoding the switch polypeptide.

In a certain embodiment, the RNA polymerase II promoter is a ubiquitous promoter.

In a particular embodiment, the ubiquitous promoter is selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In an additional embodiment, the RNA polymerase II promoter is a tissue-specific or lineage-specific promoter.

In one embodiment, the tissue-specific or lineage-specific promoter is selected from the group consisting of: a B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

In a further embodiment, the tissue-specific or lineage-specific promoter is selected from the group consisting of: an hSYN1 promoter, a TRPV1 promoter, a Naᵥ1.7 promoter, a Naᵥ1.8 promoter, and a Naᵥ1.9 promoter.

In an additional embodiment, the tissue-specific or lineage-specific promoter is a promoter specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), or the like

In a particular embodiment, the third expression cassette comprises at least one regulatory element for inducible expression that is operably linked to the polynucleotide encoding the switch polypeptide.

In one embodiment, at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

In an additional embodiment, the HSV-based vector further comprises a fourth expression cassette that comprises at least one regulatory element for inducible expression and a polynucleotide encoding a recombinase; wherein the polynucleotide encoding the CRISPR-Cas endonuclease comprises a transcription stop site flanked by one or more recombinase recognition sites.

In a particular embodiment, the one or more recombinase recognition sites selected from the group consisting of: LoxP, Lox511, Lox5171, Lox2272, m2, Lox71, Lox66, FRT, F1, F2, F3, F4, F5, FRT(LE), FRT(RE), attB, attP, attL, and attR.

In a certain embodiment, the recombinase is selected from the group consisting of: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

In one embodiment, the polynucleotide encoding the CRISPR-Cas endonuclease further encodes an inhibitory RNA and a binding site for the inhibitory RNA.

In a further embodiment, the inhibitory RNA is a miRNA or a mishRNA.

In one embodiment, the one or more guide RNAs are design to alter at least one site in a genome, optionally wherein the sequence of the one or more guide RNAs is selected from the group consisting of SEQ ID NOs: 1-55.

In a certain embodiment, the HSV-based vector further comprises a polynucleotide encoding a template for altering at least one site in a genome.

In various embodiments, an HSV-based vector as shown in any one of the figures or embodiments disclosed herein is contemplated.

In various embodiments, an HSV-based vector of any one of the preceding claims for use editing one or more genomic sequences in a cell is contemplated.

In other various embodiments, a cell comprising an HSV-based vector contemplated herein is provided.

In other various embodiments, a composition comprising an HSV-based vector or cell comprising an HSV-based vector is contemplated.

In a particular embodiment, a method of genetically modifying a cell is provided comprising introducing an HSV-based vector contemplated herein into the cell and inducing the expression of the switch polypeptide for a time sufficient to edit the genome of the cell.

In a particular embodiment, the editing of the genome in the cell comprises insertion of a regulatable transcriptional regulatory element upstream of a transcription start site in a gene of the cell.

In a certain embodiment, the transcriptional regulatory element is activated in the presence of an exogenous ligand or small molecule.

In one embodiment, the transcriptional regulatory element is activated in the absence of an exogenous ligand or small molecule.

In a further embodiment, the transcriptional regulatory element is repressed in the presence of an exogenous ligand or small molecule.

In a particular embodiment, the transcriptional regulatory element is repressed in the absence of an exogenous ligand or small molecule.

In an additional embodiment, the transcriptional regulatory element is inserted upstream of a gene associated with the regulation of pain.

In one embodiment, the transcriptional regulatory element is inserted upstream of a gene encoding a voltage gated sodium channel.

In a particular embodiment, the voltage gated sodium channel is selected from the group consisting of: Naᵥ1.1, Naᵥ1.3, Naᵥ1.6, Naᵥ1.7, Naᵥ1.8, and Naᵥ1.9, e.g. shown in Table 1.

TABLE 1
Known voltage-gated sodium channels and associated indications

| Protein | Gene | Tissue Location | Indications |
|---|---|---|---|
| Naᵥ1.1 | SCN1A | CNS, PNS, heart | Epilepsy, GEFS+, Dravet syndrome (severe |
| | | | myoclonic epilepsy of infancy or SMEI), |
| | | | borderline SMEI (SMEB), West syndrome |
| | | | (infantile spasms), Doose syndrome |
| | | | (myoclonic astatic epilepsy), intractable |
| | | | childhood epilepsy with generalized tonic- |
| | | | clonic seizures (ICEGTC), Panayiotopouos |
| | | | syndrome, familial hemiplegic migraine |
| | | | (FMH), familial autism, Rasmussen's |
| | | | encephalitis, Lennox-Gastaut syndrome |
| Naᵥ1.2 | SCN2A | CNS, PNS | Inherited febrile seizures and epilepsy |
| Naᵥ1.3 | SCN3A | CNS, PNS, heart | Epilepsy, pain |
| Naᵥ1.4 | SCN4A | skeletal muscle | Hyperkalemic periodic paralysis, paramyotonia |
| | | | congenita, and potassium-aggravated myotonia |
| Naᵥ1.5 | SCN5A | heart, skeletal | Long QT syndrome, Brugada syndrome, |
| | | muscle, smooth | idiopathic ventricular fibrillation, irritable |
| | | muscle, CNS | bowel syndrome (IBS) |
| Naᵥ1.6 | SCN8A | CNS, PNS, heart | epilepsy |
| Naᵥ1.7 | SCN9A | PNS | Erythromelalgia, paroxysmal extreme pain |
| | | | disorder (PEPD), channelopathy-associated |
| | | | insensitivity to pain (CIP), fibromyalgia |
| Naᵥ1.8 | SCN10A | PNS | Pain, neuropsychiatric disorders |
| Naᵥ1.9 | SCN11A | PNS | Pain |
| Naₓ | SCN7A | cardiac, skeletal | None known |
| | | muscle, PNS | |

CNS: Central Nervous System,
PNS: Peripheral Nervous System including Dorsal Root Ganglion (DRG) and Trigeminal Ganglion (TGG) neurons

In one embodiment, a method of using an HSV-based vector contemplated herein, a cell comprising an HSV-based vector contemplated herein, or a composition contemplated herein to treat, prevent, or ameliorate at least one symptom of a monogenetic disease, to modify checkpoint genes in T cells used for T cell therapy, to modify nociceptive genes, to modify genes in viral genomes, to modify genes involved in neurodegenerative diseases, to modify genes involved in polycystic kidney disease or Walker-Warburg syndrome, to modify genes involved in trinucleotide repeat diseases, to modify genes involved in inflammatory disease, to modify genes involved in cancer, to modify genes involved in liver disease, to modify genes involved in retinal diseases, polynucleotide sequences that contribute to aberrant splicing, and to modify genes interregulatory genes is provided.

In a certain embodiment, a method of using an HSV-based vector contemplated herein to knockout or disrupt a gene or genetic regulatory sequence encoding a checkpoint gene in a T cell is provided.

In a further embodiment, a method of using an HSV-based vector contemplated herein knockout or disrupt a gene or genetic regulatory sequence, correct a sequence in the genome, or insert genetic material into the genome to treat a hemoglobinopathy in a subject is provided.

In a particular embodiment, a method of using an HSV-based vector contemplated herein to knockout or disrupt a nociceptive gene to treat neuropathic pain is provided.

In an additional embodiment, a method of using an HSV-based vector contemplated herein to knockout or disrupt a viral gene for the destruction of latent viral genomes, e.g., from HIV, HSV, or HPV is provided.

In a particular embodiment, a method of using an HSV-based vector contemplated herein to knockout or disrupt a region of trinucleotide repeat expansion in a gene involved in neurodegenerative diseases is provided

In another embodiment, a method of using an HSV-based vector contemplated herein to knockout or disrupt a region involved in polycystic kidney disease or Walker-Warburg syndrome is provided.

In a particular embodiment, a method of using an HSV-based vector contemplated herein to knockout or disrupt CSF-1 production to deplete microglia and treat neuroinflammatory diseases such as Alzheimer's disease and Parkinson's disease is provided.

In one embodiment, a method of using an HSV-based vector contemplated herein to correct a sequence in FVIII or insert a copy of a normal copy of the FVIII gene in a cell in order to treat hemophilia is provided.

In a certain embodiment, a method of using an HSV-based vector contemplated herein to correct a sequence in the genome, or insert genetic material into the genome to treat a retinal disease, e.g, correct a rhodopsin mutation to treat retinitis pigmentosa or correct a mutation in ABCA4 to treat Stargardt disease is provided.

In one embodiment, a method of using an HSV-based vector contemplated herein to correct a sequence in the genome, or insert genetic material into the genome to treat a disease resulting from aberrant splicing is provided.

In an additional embodiment, a method of using an HSV-based vector contemplated herein to knockout or disrupt a gene or genetic regulatory sequence, correct a sequence in the genome, or insert genetic material into the genome to alter an interregulatory gene is provided.

In one embodiment, a method of using an HSV-based vector contemplated herein to treat Aicardi-Goutières Syndrome; Alexander Disease; Allan-Herndon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; or Xeroderma Pigmentosum is provided.

In one embodiment, a method of using an HSV-based vector contemplated herein to treat a monogenic disease, disorder, or condition is provided.

In a particular embodiment, the monogenic disease, disorder, or condition is 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis; anauxetic dysplasia; androgen insentivity syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT1 deficiency; glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborm errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome Imag; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1 Imag; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann corneal dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; or Zellweger disease.

In one embodiment, a method of using an HSV-based vector contemplated herein to treat macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue is provided.

In any of the foregoing embodiments or embodiments contemplated elsewhere herein, the HSV-based vector is an HSV-1 based vector.

In any of the foregoing embodiments or embodiments contemplated elsewhere herein, the HSV-based vector is an HSV-2 based vector.

### BRIEF DESCRIPTION OF THE SEVERAL VIEWS OF THE DRAWINGS

FIG. 1 shows a diagram of an HSV-based vector for the delivery of a CRISPR-Cas self-regulating genome editing platform for gene knockout.

FIG. 2 shows a diagram of an HSV-based vector for the delivery of a CRISPR-Cas self-regulating genome editing platform for genome correction or insertion.

FIG. 3 shows a diagram of an HSV-based vector for the delivery of a CRISPR-Cas self-regulating genome editing platform for gene knockouts.

FIG. 4 shows a diagram of an HSV-based vector for the delivery of a CRISPR-Cas self-regulating genome editing platform for genome correction or insertion.

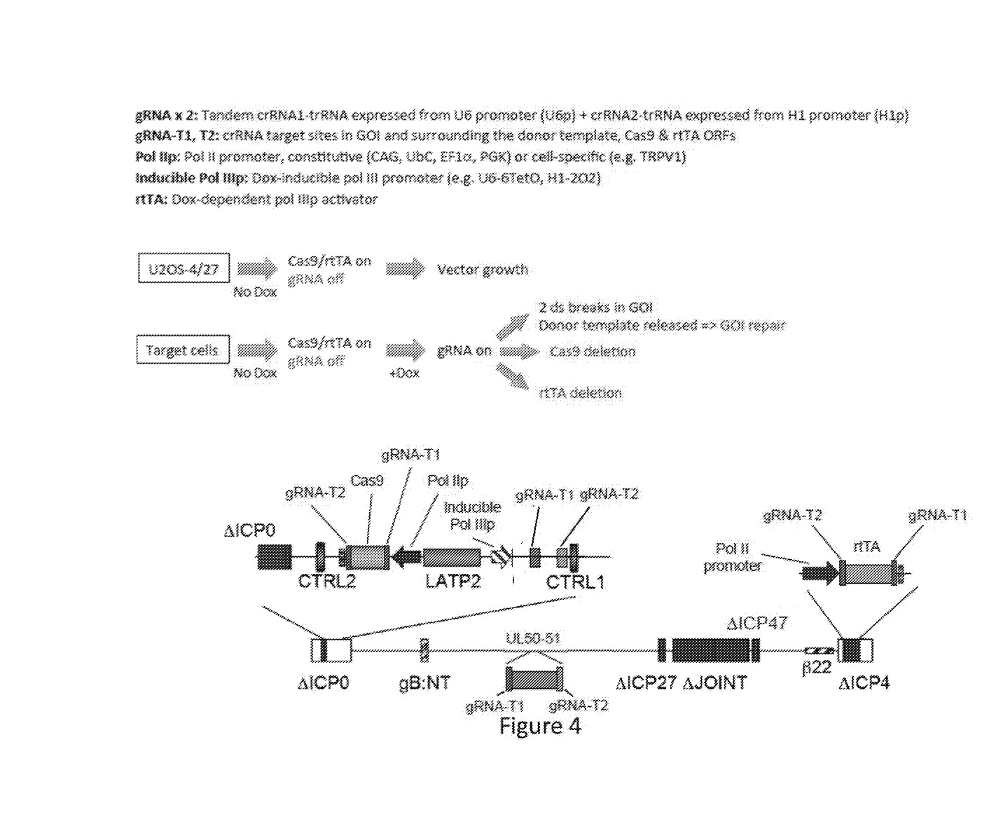

FIG. 5 shows a diagram of an HSV-based vector for the delivery of a CRISPR-Cas Cre recombinase-controlled genome editing platform for gene knockouts.

FIG. 6 shows a diagram of an HSV-based vector for the delivery of a CRISPR-Cas Cre recombinase-controlled genome editing platform for genome correction or insertion.

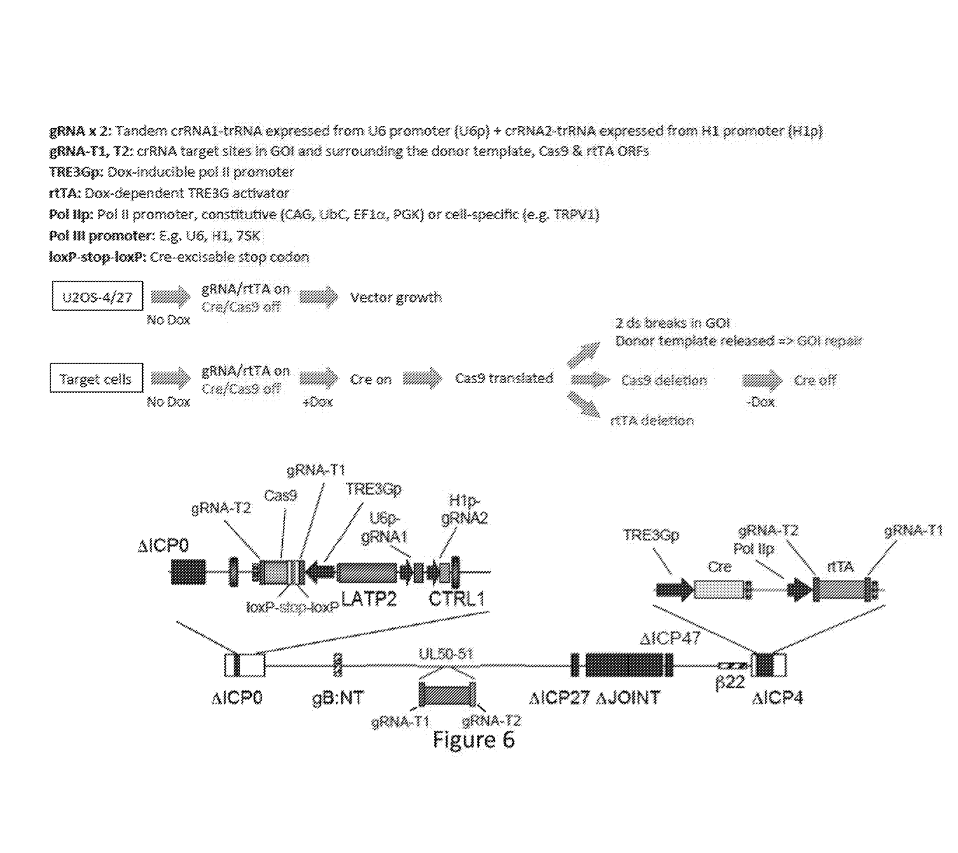

FIG. 7 shows a diagram of an HSV-based vector for the delivery of a CRISPR-Cas miRNA regulated genome editing platform for gene knockouts.

FIG. 8 shows a diagram of an HSV-based vector for the delivery of a CRISPR-Cas miRNA regulated genome editing platform for genome correction or insertion.

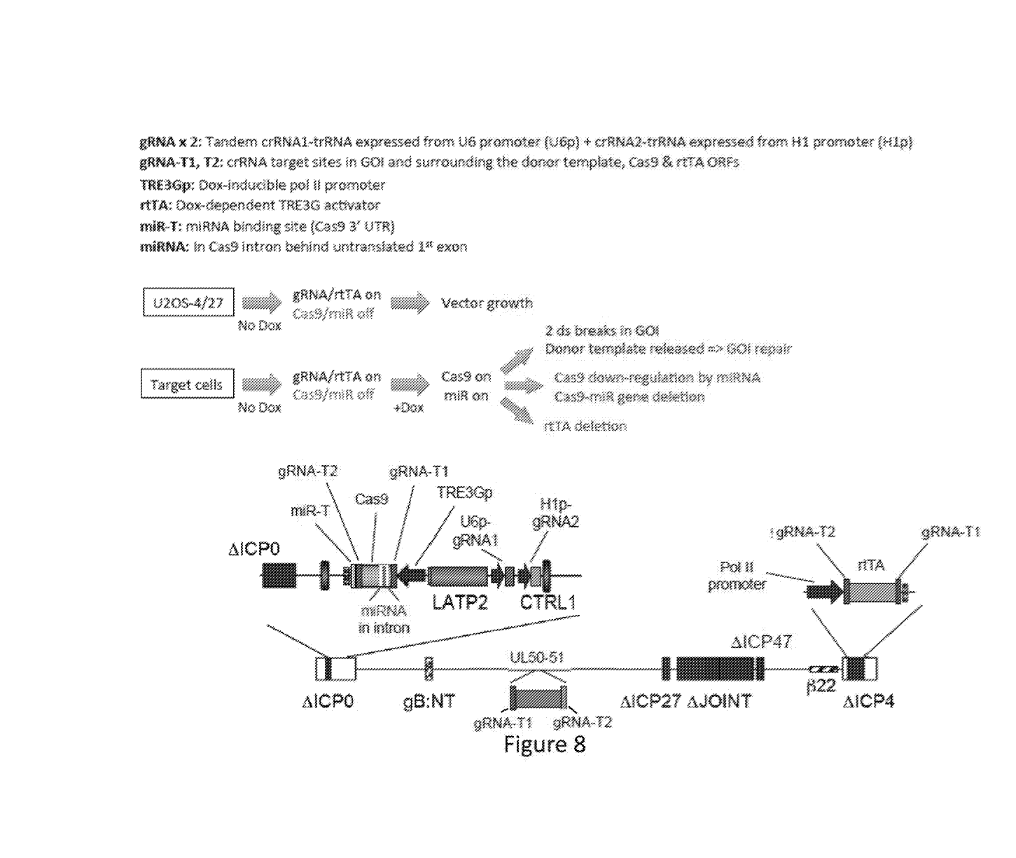

FIG. 9 shows a diagram of an HSV-based vector for the delivery of a CRISPR-Cas Cre recombinase-controlled genome editing platform for genome correction or insertion.

### BRIEF DESCRIPTION OF THE SEQUENCE IDENTIFIERS

SEQ ID NOs: 1-55 (Table 3) set forth the polynucleotide sequences of exemplary sgRNAs.

### DETAILED DESCRIPTION OF THE INVENTION

### A. Overview

The present invention generally relates to a robust genome editing platform that offers numerous advantages over existing genome editing platforms because existing platforms are rarely efficiently delivered in vivo, because existing plateforms have small payload capacities, and because existing platforms pose relatively high risks for off-target genome editing effects.

In contrast, the vectors contemplated herein have maximum payload capacities of about 150 kb, are efficiently delivered in vitro, ex vivo, or in vivo, and are engineered to provide a safe and reliable genome editing platform.

In various embodiments, an HSV-based vector comprising an inducibly and transiently regulatable genome editing platform for the disruption, deletion, correction, or insertion of genetic material at a genome sequence is provided. The HSV-vectors contemplated herein comprise a CRISPR-Cas genome editing platform that has been modified to enhance both safety and efficacy of the genome editing. Without wishing to be bound to a particular theory, the HSV-based vectors provide the advantage of delivering a complete genome editing platform in a single vector and also provide a more efficient genome editing platform that is less prone to off-target effects. The genome editing platform is less prone to off-target effects, in part, because the HSV-based vectors contemplated herein provide inducibly and/or transiently regulatable CRISPR-Cas endonucleases.

Accordingly, the present invention addresses an unmet clinical need for improving the efficacy of gene therapy in the treatment of genetic diseases. The invention relates to polynucleotides, polypeptides, vectors, genetically engineered cells, and related compositions to facilitate a desired therapeutic outcome.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. For the purposes of the present invention, the following terms are defined below.

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “isolated” means material that is substantially or essentially free from components that normally accompany it in its native state. In particular embodiments, the term “obtained” or “derived” is used synonymously with isolated.

### C. Polynucleotides

In various illustrative embodiments, the present invention contemplates, in part, polynucleotides, polynucleotides encoding polypeptides and fusion polypeptides, viral vector polynucleotides, and compositions comprising the same. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), tracrRNA, crRNA, single guide RNA (sgRNA), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxynucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

A “genomic sequence regulating transcription of” or a “genomic sequence that regulates transcription or” refers to a polynucleotide sequence that is associated with the transcription of a gene. In one embodiment, the genomic sequence regulates transcription because it is a binding site for a polypeptide that represses or decreases transcription or a polynucleotide sequence associated with a transcription factor binding site that contributes to transcriptional repression.

A “cis-acting sequence regulating transcription of” or a “cis-acting nucleotide sequence that regulates transcription or” or equivalents refers to a polynucleotide sequence that is associated with the transcription of a gene. In one embodiment, the cis-acting sequence regulates transcription because it is a binding site for a polypeptide that represses or decreases transcription or a polynucleotide sequence associated with a transcription factor binding site that contributes to transcriptional repression.

A “regulatory element” or “cis-acting sequence” or “transcriptional regulatory element” or equivalents thereof refer to an expression control sequence that comprises a polynucleotide sequence that is associated with the transcription or expression of a polynucleotide sequence encoding a polypeptide.

A “regulatory element for inducible expression” refers to a polynucleotide sequence that is a promoter, enhancer, or functional fragment thereof that is operably linked to a polynucleotide to be expressed. The regulatory element for inducible expression responds to the presence or absence of a molecule that binds the element to increase (turn-on) or decrease (turn-off) the expression of the polynucleotide operably linked thereto. Illustrative regulatory elements for inducible expression include, but are not limited to, a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

A “regulatory element for transient expression” refers to a polynucleotide sequence that can be used to briefly or temporarily express a polynucleotide nucleotide sequence. In particular embodiments, one or more regulatory elements for transient expression can be used to limit the duration of a polynucleotide. In certain embodiments, the preferred duration of polynucleotide expression is on the order of minutes, hours, or days. Illustrative regulatory elements for transient expression include, but are not limited to, nuclease target sites, recombinase recognition sites, and inhibitory RNA target sites. In addition, to some extent, in particular embodiments, a regulatory element for inducible expression may also contribute to controlling the duration of polynucleotide expression.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The terms “nucleic acid cassette” or “expression cassette” as used herein refers to polynucleotide sequences within a larger polynucleotide, such as a vector, which are sufficient to express one or more RNAs from a polynucleotide. The expressed RNAs may be translated into proteins, may function as guide RNAs or inhibitory RNAs to target other polynucleotide sequences for cleavage and/or degradation. In one embodiment, the nucleic acid cassette contains one or more polynucleotide(s)-of-interest. In another embodiment, the nucleic acid cassette contains one or more expression control sequences operably linked to one or more polynucleotide(s)-of-interest. Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, e.g., guide RNA or inhibitory RNA, as contemplated herein. In a particular embodiment, a polynucleotide-of-interest encodes a polypeptide or fusion polypeptide having one or more enzymatic activities, such as a nuclease activity and/or chromatin remodeling or epigenetic modification activities.

Vectors may comprise 1, 2, 3, 4, 5, 6, 7, 8, 9 or 10 or more nucleic acid cassettes. In a preferred embodiment of the invention, a nucleic acid cassette comprises one or more expression control sequences operably linked to a component of a genome editing platform for gene therapy. The cassette can be removed from or inserted into other polynucleotide sequences, e.g., a plasmid or viral vector, as a single unit.

In one embodiment, a polynucleotide contemplated herein comprises 1, 2, 3, 4, 5, 6, 7, 8, 9, or more nucleic acid cassettes any number or combination of which may be in the same or opposite orientations.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest encodes an inhibitory polynucleotide including, but not limited to, a crRNA, a tracrRNA, a single guide RNA (sgRNA), an siRNA, an miRNA, an shRNA, a ribozyme or another inhibitory RNA.

In one embodiment, a polynucleotide-of-interest comprises a crRNA, a tracrRNA, or a single guide RNA (sgRNA). These RNAs are part of the CRISPR (Clustered Regularly Interspaced Short Palindromic Repeats)/Cas (CRISPR Associated) nuclease system; a recently engineered nuclease system based on a bacterial system that can be used for mammalian genome engineering. See, e.g., Jinek et al. (2012) Science 337:816-821; Cong et al. (2013) Science 339:819-823; Mali et al. (2013) Science 339:823-826; Qi et al. (2013) Cell 152:1173-1183; Jinek et al. (2013), eLife 2:e00471; David Segal (2013) eLife 2:e00563; Ran et al. (2013) Nature Protocols 8(11):2281-2308; Zetsche et al. (2015) Cell 163(3):759-771; PCT Pub. Nos.: WO2007025097; WO2008021207; WO2010011961; WO2010054108; WO2010054154; WO2012054726; WO2012149470; WO2012164565; WO2013098244; WO2013126794; WO2013141680; WO2013142578; U.S. Pat. App. Pub. Nos: US20100093617; US20130011828; US20100257638; US20100076057; US20110217739; US20110300538; US20130288251; US20120277120; and U.S. Pat. No. 8,546,553, each of which is incorporated herein by reference in its entirety.

Multiple class 1 CRISPR-Cas systems, which include the type I and type III systems, have been identified and functionally characterized in detail, revealing the complex architecture and dynamics of the effector complexes (Brouns et al., 2008, Marraffini and Sontheimer, 2008, Hale et al., 2009, Sinkunas et al., 2013, Jackson et al., 2014, Mulepati et al., 2014). In addition, several class 2-type II CRISPR-Cas systems that employ homologous RNA-guided endonucleases of the Cas9 family as effectors have also been identified and experimentally characterized (Barrangou et al., 2007, Garneau et al., 2010, Deltcheva et al., 2011, Sapranauskas et al., 2011, Jinek et al., 2012, Gasiunas et al., 2012). A second, putative class 2-type V CRISPR-Cas system has been recently identified in several bacterial genomes. The putative type V CRISPR-Cas systems contain a large, ˜1,300 amino acid protein called Cpf1 (CRISPR from Prevotella and Francisella 1).

The CRISPR/Cas nuclease system can be used to introduce a double-strand break in a target polynucleotide sequence, which may be repaired by non-homologous end joining (NHEJ) in the absence of a polynucleotide template, e.g., a DNA template for altering at least one site in a genome, or by homology directed repair (HDR), i.e., homologous recombination, in the presence of a polynucleotide repair template. Cas9 and Cpf1 nucleases can also be engineered as nickases, which generate single-stranded DNA breaks that can be repaired using the cell's base-excision-repair (BER) machinery or homologous recombination in the presence of a repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence flanked on either side by sequences bearing homology to the target.

In various embodiments, vectors contemplated herein contain polynucleotides to be expressed that are flanked by one or more crRNA or sgRNA target sites to transiently regulate the expression of the polynucleotide.

In one embodiment, wherein a crRNA or sgRNA is directed against a polynucleotide sequence encoding a polypeptide, NHEJ of the ends of the cleaved genomic sequence may result in a normal polypeptide, a loss-of- or gain-of-function polypeptide, or knock-out of a functional polypeptide.

In another embodiment, wherein a crRNA or sgRNA is directed against a polynucleotide sequence encoding a cis-acting sequence that regulates mRNA expression of a polynucleotide sequence encoding a polypeptide, NHEJ of the genomic sequence may result increased expression, decreased expression, or complete loss of expression of the mRNA and polypeptide.

In another embodiment, wherein a polynucleotide template for repair of the cleaved genomic sequence is provided, the genomic locus is repaired with the sequence of the template by homologous recombination. In one embodiment, the repair template comprises a polynucleotide sequence that is different from a targeted genomic sequence. In one embodiment, the repair template comprises one or more polynucleotides that restores function of the targeted genomic sequence or restores the natural polynucleotide sequence encoding a wild type allele of a polypeptide. In another embodiment, the repair DNA template comprises one or more polynucleotides that reduces or eliminates function of the targeted genomic sequence or decreases the expression of the natural polynucleotide sequence encoding a wild type allele of a polypeptide and/or increasing the expression of a variant polypeptide.

As used herein, the term “guide RNA” refers to a “crRNA” and/or an “sgRNA.”

As used herein, the term “crRNA” refers to an RNA comprising a region of partial or total complementarity referred to herein as a “spacer motif” to a target polynucleotide sequence referred to herein as a protospacer motif. In one embodiment, a protospacer motif is a 20 nucleotide target sequence. In particular embodiments, the protospacer motif is 18, 19, 20, 21, 22, 23, 24, 25, 26, 27, 28, 29, 30, or more nucleotides. Without wishing to be bound by any particular theory, it is contemplated that protospacer target sequences of various lengths will be recognized by different bacterial species.

In one embodiment, the region of complementarity comprises a polynucleotide sequence that is at least 80%, 81%, 82%, 83%, 84%, 85%, 86%, 8′7%, 88%, 89%, 90%, 91%, 92%, 93%, 94%, 95%, 96%, 97%, 98%, 99% or 100% identical to the protospacer sequence. In a related embodiment, at least 10, 11, 12, 13, 14, 15, 16, 17, 18, 19, or 20 or more polynucleotides in the region of complementarity are identical to the protospacer motif. In a preferred embodiment, at least 10 of the 3′ most sequence in the protospacer motif is complementary to the crRNA sequence.

As used herein, the term “tracrRNA” refers to a trans-activating RNA that associates with the crRNA sequence through a region of partial complementarity and serves to recruit a Cas9 nuclease to the protospacer motif. In one embodiment, the tracrRNA is at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In one embodiment, the tracrRNA is about 85 nucleotides in length.

In one embodiment, the crRNA and tracrRNA are engineered into one polynucleotide sequence referred to herein as a “single guide RNA” or “sgRNA.” The crRNA equivalent portion of the sgRNA is engineered to guide the Cas9 nuclease to target any desired protospacer motif. In one embodiment, the tracrRNA equivalent portion of the sgRNA is engineered to be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. Illustrative examples of sgRNAs suitable for use in particular embodiments contemplated herein include, but are not limited to sgRNA sequences as set forth in SEQ ID NOs: 1-55 (see Table 3).

The protospacer motif abuts a short protospacer adjacent motif (PAM), which plays a role in recruiting a Cas9/RNA or Cpf1/RNAcomplex. Cas9 polypeptides recognize PAM motifs specific to the Cas9 polypeptide. Accordingly, the CRISPR/Cas9 system can be used to target and cleave either or both strands of a double-stranded polynucleotide sequence flanked by particular 3′ PAM sequences specific to a particular Cas9 polypeptide. PAMs may be identified using bioinformatics or using experimental approaches. Esvelt et al., 2013, Nature Methods. 10(11):1116-1121, which is hereby incorporated by reference in its entirety.

In one embodiment, a polynucleotide encodes a transiently regulatable Cas9 polypeptide. In one embodiment, the polynucleotide comprises a regulatory element for transient expression of and a polynucleotide encoding a Cas9 polypeptide. A Cas9 polypeptide can be engineered as a double-stranded DNA endonuclease or a nickase or catalytically dead Cas9, and forms a ternary target complex with a crRNA and a tracrRNA for site specific DNA recognition and cleavage if catalytically active. Normally, tracrRNA is involved in the maturation of precursor crRNA. Following co-processing of tracrRNA and pre-crRNA by RNase III, a dual-tracrRNA:crRNA guides the CRISPR-associated endonuclease Cas9 to site-specifically cleave a target DNA, e.g., protospacer sequence.

Unlike Cas9 systems, Cpf1-containing CRISPR-Cas systems have three features. First, Cpf1-associated CRISPR arrays are processed into mature crRNAs without the requirement of an additional trans-activating crRNA (tracrRNA) (Deltcheva et al., 2011, Chylinski et al., 2013). Second, Cpf1-crRNA complexes efficiently cleave target DNA proceeded by a short T-rich protospacer-adjacent motif (5′-TTN PAM), in contrast to the G-rich PAM following the target DNA for Cas9 systems. Third, Cpf1 introduces a staggered DNA double-stranded break with a 4 or 5-nt 5′ overhang.

In one embodiment, a polynucleotide encodes a transiently regulatable Cpf1 polypeptide. In one embodiment, the polynucleotide comprises a regulatory element for transient expression of and a polynucleotide encoding a Cpf1 polypeptide. A Cpf1 polypeptide can be engineered as a double-stranded DNA endonuclease or a nickase or catalytically dead Cpf1, and forms a target complex with a crRNA for site specific DNA recognition and cleavage if catalytically active. Following processing of pre-crRNA by RNase III, a crRNA guides the CRISPR-associated endonuclease Cpf1 to site-specifically cleave a target DNA, e.g., protospacer sequence.

In one embodiment, one or more crRNAs or sgRNAs contemplated herein, can be designed to target a polynucleotide sequence involved in the pathogenesis of a monogenetic disease, checkpoint genes in T cells used for T cell therapy, nociceptive genes, genes in viral genomes, genes involved in neurodegenerative diseases, genes involved in polycystic kidney disease, Walker-Warburg syndrome, genes involved in trinucleotide repeat diseases, genes involved in inflammatory disease, genes involved in cancer, genes involved in liver disease, genes involved in retinal diseases, polynucleotide sequences that contribute to aberrant splicing, and interregulatory genes.

In one embodiment, the one or more crRNAs comprises a pair of offset crRNAs complementary to opposite strands of the target site. In one embodiment, the one or more sgRNAs comprises a pair of offset sgRNAs complementary to opposite strands of the target site. Without wishing to be bound by any particular theory, in some embodiments, it is contemplated that using a pair of offset crRNAs or sgRNAs with a Cas9 or Cpf1 nickase contemplated herein reduces off target genome editing. A single nick is repaired efficiently using a cell's base-excision-repair (BER) machinery. Thus, a large majority of single nicks do not result in nonhomologous end joining (NHEJ)-mediated indels. By inducing offset nicks, off-target single nick events will likely result in very low indel rates.

In one embodiment, offset nicks are induced using a pair of offset crRNAs or sgRNAs with a Cas9 or Cpf1 nickase increases site-specific NHEJ or HDR (when a repair template is provided). In one embodiment, a pair of offset crRNAs or sgRNAs is designed to create 5′ overhangs via the offset nicks to increase the rate of site-specific NHEJ or homologous recombination.

In one embodiment, the pair of offset crRNAs or sgRNAs are offset by at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, or at least 100 nucleotides.

In one embodiment, the pair of offset crRNAs or sgRNAs are offset by about 5 to about 100 nucleotides, about 10 to about 50 nucleotides, about 10 to about 40 nucleotides, about 10 to about 30 nucleotides, about 10 to about 20 nucleotides, or about 15 to 30 nucleotides, as well as all intermediate lengths or ranges.

In one embodiment, a crRNA or sgRNA is designed to induce a single nick with a Cas9 or Cpf1 nickase; in combination with a double-stranded or single-stranded repair template polynucleotide, the nick is repaired using homologous recombination with minimal off-target indel effects.

Illustrative examples for bacterial sources of Cas9 polynucleotides encoding a Cas9 polypeptide suitable for use in the methods contemplated herein and corresponding PAM motifs include, but are not limited to: Staphylococcus aureus, (NNGRR), Streptococcus pyogenes Cas9 (NGG); Streptococcus thermophilis Cas9 (NNNNGANN, NNNNGTTN, NNNNGNNT, NNAGAAW, NNNNGTNN, NNNNGNTN); Treponema denticola Cas9 (NAAAAN, NAAANC, NANAAC, NNAAAC); and Neisseria meningitidis Cas9 (NNAGAA, NNAGGA, NNGGAA, NNNNGATT, NNANAA, NNGGGA). Without wishing to be bound to any particular theory, a virtually limitless selection of protospacer motifs may be targeted using the CRISPR technology because a suitable Cas9 may be selected to target any protospacer based on the sequence of the adjacent PAM motif.

Illustrative examples for bacterial sources of Cpf1 polynucleotides encoding a Cpf1 polypeptide suitable for use in the methods contemplated herein include, but are not limited to: Francisella novicida, Acidaminococcus sp. BV3L6, and Lachnospiraceae bacterium ND2006.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from ˜70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts or “mishRNA.” This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

In one embodiment, a polynucleotide encoding a CRISPR-Cas endonuclease comprises an intron that comprises a miRNA and a 3′UTR that comprises a corresponding miRNA target site. Without wishing to be bound to any particular theory, it is contemplated that this architecture can be used to transiently regulate the expression of the CRISPR-Cas endonuclease and minimize the off-target effects of the endonuclease either alone or in combination with one or more additional regulatory elements to regulate the transient expression of the endonuclease.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

In one embodiment, an expression cassette comprises one or more of a crRNA, a tracrRNA, sgRNA, an siRNA, an miRNA, an shRNA, or a ribozyme and further comprises one or more regulatory sequences, such as, for example, a strong constitutive RNA pol III promoter, e.g., human or mouse U6 snRNA promoter, the human and mouse H1 RNA promoter, or the human tRNA-val promoter; an inducible RNA pol III promoter, e.g., U6-6TetO promoter, H1-peroxide promoter; or a strong constitutive or inducible RNA pol II promoter, as described elsewhere herein.

The polynucleotides contemplated herein, regardless of the length of the coding sequence itself, may be combined with other DNA sequences, such as expression control sequences, regulatory elements, promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), guide RNA target sites, termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well established techniques known and available in the art. In order to express a desired polypeptide, a nucleotide sequence encoding the polypeptide, can be inserted into appropriate vector, such as a viral vector. In preferred embodiments, the viral vector is a herpesvirus viral vector, e.g., herpes simplex virus.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity.

Depending on the vector system and host utilized, any number of suitable transcription and translation elements, including ubiquitous promoters and inducible promoters may be used.

In particular embodiments, a polynucleotide for use in practicing the invention is a vector, including but not limited to expression vectors and viral vectors, and includes exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In particular embodiments, the vector comprises one or more RNA pol II and/or RNA pol III promoters.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) or regulatory element and a second polynucleotide sequence, e.g., a polynucleotide-of-interest, wherein the expression control sequence or regulatory element directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In a particular embodiment, it may be desirable to use a tissue-specific promoter to achieve cell type specific, lineage specific, or tissue-specific expression of a desired polynucleotide sequence. Illustrative examples of tissue specific promoters include, but are not limited to: an B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

According to certain embodiments, the cell type specific promoter is specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), etc.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In particular embodiments, polynucleotides contemplated herein comprise at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. As used herein, the terms “recombination sequence,” “recombination site,” or “site specific recombination site” refer to a particular nucleic acid sequence to which a recombinase recognizes and binds.

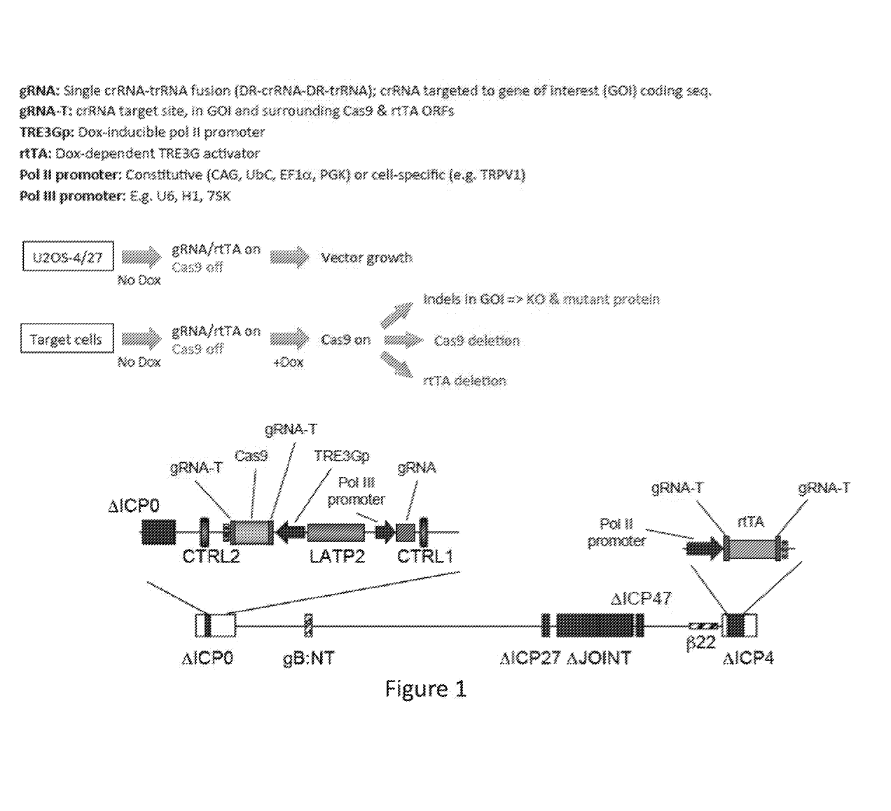

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagaraj an et al., 2001; Belteki et al., 2003). Thus, typical strategies position by homologous recombination an attP-bearing “docking site” into a defined locus, which is then partnered with an attB-bearing incoming sequence for insertion.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, a polynucleotide encoding a polypeptide comprises a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 56), where R is a purine (A or G) (Kozak, 1986. Cell. 44(2):283-92, and Kozak, 1987. Nucleic Acids Res. 15(20):8125-48).

In particular embodiments, polynucleotides comprise a polyadenylation sequence 3′ of a polynucleotide encoding a polypeptide to be expressed. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments the poly(A) sequence is an SV40 polyA sequence, a bovine growth hormone polyA sequence (BGHpA), a rabbit β-globin polyA sequence (rβgpA), or another suitable heterologous or endogenous polyA sequence known in the art.

### D. Polypeptides

The present invention contemplates, in part, compositions comprising polypeptides, fusion polypeptides, and vectors that express polypeptides.

“Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated peptide” or an “isolated polypeptide” and the like, as used herein, refer to in vitro isolation, purification, recombinant production, or synthesis of a peptide or polypeptide molecule from a cellular environment, and from association with other components of the cell, i.e., it is not significantly associated with in vivo substances.

Polypeptides include biologically active “polypeptide fragments.” As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. Polypeptide fragments refer to a polypeptide, which can be monomeric or multimeric, that has an amino-terminal deletion, a carboxyl-terminal deletion, and/or an internal deletion or substitution of one or more amino acids of a naturally-occurring or recombinantly-produced polypeptide. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a polypeptide or the binding or cleavage specificity of a Cas or Cpf1 polypeptide by introducing one or more substitutions, deletions, additions and/or insertions into the polypeptide. Preferably, polypeptides of the invention include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity thereto.

As noted above, polypeptides contemplated herein may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant polypeptide of the invention, one skilled in the art, for example, can change one or more of the codons of the encoding DNA sequence, e.g., according to Table 2.

TABLE 2
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Exemplary conservative substitutions are described in U.S. Provisional Patent Application No. 61/241,647, the disclosure of which is herein incorporated by reference.

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In various embodiments, Cas9 polypeptides are contemplated. Cas9 is the signature protein characteristic for type II CRISPR nuclease systems in bacteria. At least 235 Cas9 orthologs have been identified in 203 bacterial species, the names and sequences of which are herein incorporated by reference in their entirety from the publication and supplemental information of Chylinski et al., 2013. RNA Biol. 10(5): 726-737. Conserved regions of Cas9 orthologs include a central HNH endonuclease domain and a split RuvC/RNase H domain.

In particular embodiments, a suitable Cas9 polypeptide sequence may be obtained from the following illustrative list of bacterial species: Staphylococcus aureus, Enterococcus faecium, Enterococcus italicus, Listeria innocua, Listeria monocytogenes, Listeria seeligeri, Listeria ivanovii, Streptococcus agalactiae, Streptococcus anginosus, Streptococcus bovis, Streptococcus dysgalactiae, Streptococcus equinus, Streptococcus gallolyticus, Streptococcus macacae, Streptococcus mutans, Streptococcus pseudoporcinus, Streptococcus pyogenes, Streptococcus thermophilus, Streptococcus gordonii, Streptococcus infantarius, Streptococcus macedonicus, Streptococcus mitis, Streptococcus pasteurianus, Streptococcus suis, Streptococcus vestibularis, Streptococcus sanguinis, Streptococcus downei, Neisseria bacilliformis, Neisseria cinerea, Neisseria flavescens, Neisseria lactamica, Neisseria meningitidis, Neisseria subflava, Lactobacillus brevis, Lactobacillus buchneri, Lactobacillus casei, Lactobacillus paracasei, Lactobacillus fermentum, Lactobacillus gasseri, Lactobacillus jensenii, Lactobacillus johnsonii, Lactobacillus rhamnosus, Lactobacillus ruminis, Lactobacillus salivarius, Lactobacillus sanfranciscensis, Corynebacterium accolens, Corynebacterium diphtherias, Corynebacterium matruchotii, Campylobacter jejuni, Clostridium perfringens, Treponema vincentii, Treponema phagedenis, and Treponema denticola.

Cas9 polypeptides target double-stranded polynucleotide sequences flanked by particular 3′ PAM sequences specific to a particular Cas9 polypeptide. Each Cas9 nuclease domain cleaves one DNA strand. Cas9 polypeptides naturally contain domains homologous to both HNH and RuvC endonucleases. The HNH and RuvC-like domains are each responsible for cleaving one strand of the double-stranded DNA target sequence. The HNH domain of the Cas9 polypeptide cleaves the DNA strand complementary to the tracrRNA:crRNA or sgRNA. The RuvC-like domain of the Cas9 polypeptide cleaves the DNA strand that is not-complementary to the tracrRNA:crRNA or sgRNA.

In one embodiment, a Cas9 polypeptide or biologically active fragment thereof comprising catalytic activity of the HNH and RuvC domains is contemplated.

In particular embodiments, a Cas9 polypeptide variant is contemplated comprising one or more amino acids additions, deletions, mutations, or substitutions in the HNH or RuvC-like endonuclease domains that decreases or eliminates the nuclease activity of the variant domain. In one embodiment, the variant is a Cas9 nickase.

In one embodiment, the Cas9 polypeptide is catalytically inactive, meaning that one or more amino acids additions, deletions, mutations, or substitutions in the HNH and the RuvC-like endonuclease domains have been made to render the Cas9 catalytically inactive.

In one embodiment, a Cas9 polypeptide comprises one or more amino acids additions, deletions, mutations, or substitutions that decrease or eliminate the nuclease activity in the HNH domain. Illustrative examples of Cas9 enzymes and corresponding mutations that decrease or eliminate the nuclease activity in the HNH domain include, but are not limited to: S. aureus (D10A), S. pyogenes (D10A); S. thermophilis (D9A); T. denticola (D13A); and N. meningitidis (D16A).

In one embodiment, a Cas9 polypeptide comprises one or more amino acids additions, deletions, mutations, or substitutions that decrease or eliminate the nuclease activity in the RuvC-like domain. Illustrative examples of Cas9 enzymes and corresponding mutations that decrease or eliminate the nuclease activity in the RuvC-like domain include, but are not limited to: S. aureus (N580A), S. pyogenes (D839A, H840A, or N863A); S. thermophilis (D598A, H599A, or N622A); T. denticola (D878A, H879A, or N902A); and N. meningitidis (D587A, H588A, or N611A).

In one embodiment, a Cas9 nickase and one or more guide RNAs comprising a pair of offset guide RNAs complementary to opposite strands of the target site are used to engineer a double-strand break. In one embodiment, a pair of offset guide RNAs is designed to create 5′ overhangs via the offset nicks to increase the rate of site-specific NHEJ or homologous recombination when a DNA repair template is present.

In one embodiment, a Cas9 nickase and a guide RNA designed against a target sequence is used to engineer a single-strand break. In one embodiment, a guide RNA in combination with a Cas9 nickase and a double-stranded or single-stranded repair template is used to engineer a single-strand break that is repaired using homologous recombination with minimal off-target effects.

In various embodiments, Cpf1 polypeptides are contemplated.

In particular embodiments, a suitable Cpf1 polypeptide sequence may be obtained from the following illustrative list of bacterial species: Francisella novicida, Acidaminococcus sp. BV3L6, or Lachnospiraceae bacterium ND2006.

In one embodiment, a Cpf1 polypeptide comprises one or more amino acids additions, deletions, mutations, or substitutions that decrease or eliminate the nuclease activity in the RuvC-like domain.

Illustrative examples of Cpf1 enzymes and corresponding mutations that decrease or eliminate the nuclease activity in the RuvC-like domain include, but are not limited to: Cpf1 from Francisella novicida, wherein the mutation is a D917A, E1006A, or D1225A mutation. Polypeptides of the present invention include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

Fusion polypeptides can comprise one or more polypeptide domains or segments including, but are not limited to cell permeable peptide domains (CPP), Zn-finger DNA binding domains, nuclease domains, chromatin remodeling domains, histone modifying domains, and epigenetic modifying domains, epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In various embodiments, a fusion polypeptide comprising a Cas9 or Cpf1 endonuclease, nickase, or catalytically inactive mutant is contemplated. In one embodiment, a fusion polypeptide comprises a catalytically inactive Cas9 or Cpf1 polypeptide and a nuclease domain. In particular embodiments, the fusion polypeptide comprises an endonuclease domain that is a cleavage half-domain, such as, for example, the cleavage domain of a Type IIs restriction endonuclease such as FokI. A pair of such nuclease half-domain fusions is used for targeted cleavage for each strand of the target

In various embodiments, the fusion polypeptide or a switch fusion polypeptide comprises one or more functional domains selected from the group consisting of: a histone methylase or demethylase domains, a histone acetylase or deacetylase domains, a SUMOylation domain, an ubiquitylation or deubiquitylation domain, a DNA methylase or DNA demethylase domain, and a nuclease domain.

In one embodiment, the nuclease domain is a FOK I cleavage domain.

In one embodiment, the nuclease domain is a TREX2 domain.

Fusion polypeptides may optionally comprise a linker that can be used to link the one or more polypeptides. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: DGGGS (SEQ ID NO: 57); TGEKP (SEQ ID NO: 58) (see, e.g., Liu et al., PNAS 5525-5530 (1997)); GGRR (SEQ ID NO: 59) (Pomerantz et al. 1995, supra); (GGGGS)ₙ (SEQ ID NO: 60) (Kim et al., PNAS 93, 1156-1160 (1996.); EGKSSGSGSESKVD (SEQ ID NO: 61) (Chaudhary et al., 1990, Proc. Natl. Acad. Sci. U.S.A. 87:1066-1070); KESGSVSSEQLAQFRSLD (SEQ ID NO: 62) (Bird et al., 1988, Science 242:423-426), GGRRGGGS (SEQ ID NO: 63); LRQRDGERP (SEQ ID NO: 64); LRQKDGGGSERP (SEQ ID NO: 65); LRQKd(GGGS)₂ERP (SEQ ID NO: 66). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 67), for example, ENLYFQG (SEQ ID NO: 68) and ENLYFQS (SEQ ID NO: 69), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide. In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

In various embodiments, the fusion polypeptide or a switch fusion polypeptide is fused to a self-cleaving viral peptide and a TREX2 polypeptide to facilitate homologous recombination.

### E. Viral Vectors

In various embodiments, a vector comprises a polynucleotide sequence contemplated herein. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include Herpesvirus vectors.

In preferred embodiments, the Herpesvirus is a Herpes simplex virus (HSV).

In particular preferred embodiments, the HSV is HSV-1 or HSV-2.

In various preferred embodiments, the HSV is HSV-1.

An “HSV-based vector” is a Herpes simplex virus that has been altered to render it suitable for delivering a genome editing platform to a cell. Without wishing to be bound by any particular theory, an HSV-based viral vector is advantageous in particular embodiments herein because the HSV based viral vector can be engineered to express a plurality of expression cassettes necessary to reconstitute an entire gene editing platform in a single vector; because the HSV based viral vector can support large payloads; and because the HSV-based viral vector can be engineered for efficient delivery to a variety of cell types in vitro, in vivo, or ex vivo to provide gene therapy.

The mature HSV virion consists of an enveloped icosahedral capsid with a viral genome consisting of a linear double-stranded DNA molecule that is 152 kb and encodes approximately 84 genes. The linear double-stranded genome is composed of a long (UL) and short (US) genomic segment that contain both essential and non-essential genes. In general, accessory genes can be individually deleted without substantially compromising virus replication in standard cell cultures. By contrast, deletion of any essential gene completely blocks productive virus infection. Each genomic segment is flanked by inverted repeats creating an internal region referred to as the joint. Several genes that regulate virus replication are located in repeat regions and are therefore diploid. Consequently, the approximately 19 kb joint region can be deleted without substantially compromising virus replication, creating a large space for insertion of one or more expression cassettes. The manipulation of particular viral genes has led to the creation of three types of HSV-based vectors: amplicon, replication-defective, and replication-competent vectors.

In one embodiment, an HSV-based vector is deficient in at least one essential HSV gene. The HSV-based vector may also comprise one or more deletions of non-essential genes.

In a preferred embodiment, the HSV-based vector is replication-deficient. Most replication-deficient HSV-based vectors contain a deletion to remove one or more intermediate-early, early, or late HSV genes to prevent replication. In one embodiment, the HSV-based vector is deficient in an immediate early gene selected from the group consisting of: ICP0, ICP4, ICP22, ICP27, ICP47, and a combination thereof. In preferred embodiment, the HSV-based vector is deficient for ICP0, ICP4, ICP22, ICP27, and ICP47.

HSV-based vectors and methods for their construction are described in, for example, U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties. The sequence of HSV is published (NCBI Accession No. NC_001806; see also McGoech et al., J. Gen. Virol, 69 (PT 7), 1531-1574 (1988)), which may facilitate the generation of desired mutations in designing HSV-based vectors.

As will be evident to one of skill in the art, the term “viral vector” is widely used to refer either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. The term “HSV-based vector” or “HSV-based vector” refers to a viral vector or plasmid containing structural and functional genetic elements, or portions thereof, that are primarily derived from a Herpes simplex virus.

In further embodiments, an HSV-based vector contemplated herein may comprise one or more insulator elements flanking an expression cassette inserted into the vector. The insulator elements may be naturally occurring in an HSV, e.g., CTRL1 and CTRL2, or may be heterologous. Suitable insulators for use in particular embodiments include, but are not limited to, the chicken β-globin insulator (see Chung et al., 1993. Cell 74:505; Chung et al., 1997. PNAS 94:575; and Bell et al., 1999. Cell 98:387, incorporated by reference herein). Examples of insulator elements include, but are not limited to, an insulator from an β-globin locus, such as chicken HS4.

In various embodiments, an HSV-based vector comprising an inducibly and transiently regulatable gene editing system is provided. The HSV-based vectors comprise one or more expression cassettes that together constitute a genome editing platform for gene therapy. In a preferred embodiment, the genome editing system is a CRISPR-Cas gene editing system. The components of the CRISPR-Cas system may be inserted into one or more expression cassettes which are in turn engineered into the HSV-based vector.

In particular embodiments, the HSV-based vector is deficient in one or more genes selected from the group consisting of: ICP0, ICP4, ICP22, ICP27, ICP47, and in some embodiments, preferably deficient for ICP0, ICP4, ICP22, ICP27, and ICP47.

The expression cassettes and/or donor templates that constitute the genome editing platform may be inserted into one or more of the deficient loci for ICP0, ICP4, ICP22, ICP27, ICP47, the joint region, or into loci made deficient for non-essential genes or in intergenic regions between non-essential genes or flanking non-essential genes. Illustrative examples of HSV genes that can be replaced, engineered to contain, or that are flanked by expression cassettes and/or donor templates constituting the genome editing platforms contemplated herein include, but are not limited to ICP0, ICP4, ICP22, ICP27, ICP47, UL2, UL3, UL4, UL10, UL11, UL13, UL16, UL20, UL21, UL23, UL24, UL39, UL40, UL41, UL43, UL44, UL45, UL46, UL47, UL50, UL51, UL55, UL56, LAT, ICP34.5, US1, US2, US3, US4, US5, US7, USB, US9, US10, and US11.

In preferred embodiments, the HSV-based vectors provide the advantage of delivering a complete genome editing platform in a single vector and also provide a more efficient genome editing platform that is less prone to off-target effects. Without wishing to be bound to a particular theory, the HSV-based vectors contemplated herein provide inducibly and/or transiently regulatable CRISPR-Cas endonucleases (e.g., Cas9, Cpf1) to reduce off-target effects. In particular embodiments, the CRISPR-Cas endonuclease is transiently expressed on the order of minute, hours, or days. In particular embodiments, the CRISPR-Cas endonuclease is transiently expressed for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes or more. In certain embodiments, the CRISPR-Cas endonuclease is transiently expressed for about 1, 2, 3, 4, 5, 6, 7, 8, 9, 10, 11, or 12 hours.

In particular embodiments, an HSV-based vector comprises a genome editing platform for knocking out genes or altering the activity of cis-acting genetic regulatory elements in the genome.

In particular embodiments, an HSV-based vector comprises a genome editing platform for making corrections to the genome or inserting genetic material into the genome.

In various embodiments, an HSV-based vector comprises one or a plurality of expression cassettes encoding a transiently and inducibly regulatable CRISPR-Cas endonuclease, a polynucleotide encoding one or more guide RNAs, a polynucleotide encoding a switch polypeptide that induces expression of the CRISPR-Cas endonuclease and/or the guide RNAs, and optionally a DNA template for altering the genome. The HSV-vector may be transiently regulated by flanking the CRISPR-Cas endonuclease and/or the switch polypeptide with guide RNA target sites that match the genome target sites or recombinase recognition sites, thereby inactivating the HSV-based vector and accomplishing the desired genome editing strategy. For example, an HSV-based vector may comprise a self-inactivating nuclease construct that comprises or consists of a CRISPR-Cas endonuclease, a gRNA molecule for targeting, and flanking sites targeted by that gRNA. In this construct, the CRISPR-Cas endonuclease cleaves both the target locus and its own coding construct, thus limiting or eliminating its own expression. See, e.g., Epstein et al. “119-Engineering a Self-Inactivating CRISPR System for AAV Vectors,” American Society of Gene & Cell Therapy, 19^{th} Annual Meeting, Washington, D.C., May 4-7, 2016, specifically incorporated herein by reference in its entirety. Additional examples of self-inactivating CRISPR-Cas endonuclease constructs are provided in WO 2015/089351 A1, specifically incorporated herein by reference in its entirety. In some embodiments, a CRISPR-Cas endonuclease inactivates a co-expressed gene in the vector, but not the CRISPR-Cas endonuclease itself. See, e.g., Moore et al. Nucleic Acids Res. 43(2): 1297-1303 (2015), specifically incorporated herein by reference in its entirety. Thus, the genome editing platform and related HSV-based vectors contemplated herein provide a quantum leap in genome editing safety compared to existing strategies.

In one embodiment, the HSV-based vector comprises a first expression cassette that has at least one regulatory element for inducible expression and at least one regulatory element for transient expression operably linked to a polynucleotide encoding a CRISPR-Cas endonuclease; a second expression cassette that has an RNA pol III promoter operably linked to a polynucleotide encoding one or more guide RNAs; and a third expression cassette that has an RNA pol II promoter operably linked to a polynucleotide encoding a switch polypeptide that binds to the at least one element for inducible expression.

In one embodiment, one or more crRNAs or sgRNAs contemplated herein, can be designed to target guide RNA target sites in the HSV-based vector as well as a polynucleotide sequence involved in the pathogenesis of a monogenetic disease, checkpoint genes in T cells used for T cell therapy, nociceptive genes, genes in viral genomes, genes involved in neurodegenerative diseases, genes involved in polycystic kidney disease, Walker-Warburg syndrome, genes involved in trinucleotide repeat diseases, genes involved in inflammatory disease, genes involved in cancer, genes involved in liver disease, genes involved in retinal diseases, polynucleotide sequences that contribute to aberrant splicing, and interregulatory genes.

In one embodiment, the HSV-based vector comprises a first expression cassette that has an RNA pol II promoter and at least one regulatory element for transient expression operably linked to a polynucleotide encoding a CRISPR-Cas endonuclease; a second expression cassette that has an RNA pol III promoter and at least one regulatory element for inducible expression operably linked to a polynucleotide encoding one or more guide RNAs; and a third expression cassette that has an RNA pol II promoter operably linked to a polynucleotide encoding a switch polypeptide that binds to the at least one element for inducible expression.

In one embodiment, the HSV-based vector has a third expression cassette that has at least one regulatory element for inducible expression operably linked to a recombinase and an RNA pol II promoter operably linked to a polynucleotide encoding a switch polypeptide that binds to the at least one element for inducible expression; and the vector also has a transcription stop site flanked by the recognition sites of the recombinase in the polynucleotide encoding the CRISPR-Cas endonuclease or recombinase sites flanking the polynucleotide encoding the CRISPR-Case endonuclease, as an added layer of control of CRISPR-Cas endonuclease expression.

Illustrative examples of recombinases suitable for use in particular embodiments include, but are not limited to Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

Illustrative examples of recombinase recognition sites include, but are not limited to LoxP, Lox511, Lox5171, Lox2272, m2, Lox71, Lox66, FRT, F1, F2, F3, F4, F5, FRT(LE), FRT(RE), attB, attP, attL, and attR.

In one embodiment, the HSV-based vector has a polynucleotide encoding the CRISPR-Cas endonuclease that also encodes an inhibitory RNA and contains a recognition site for the inhibitory RNA, which provides the vector with yet another layer of control of CRISPR-Cas endonuclease expression.

In particular embodiments, the Cas9, Cpf1, and/or switch polypeptides are fusion polypeptides, optionally fused to a nuclease domain, including, without limitation, a FOK I nuclease domain or a TREX2 domain.

In other particular embodiments, the Cas9, Cpf1, and/or switch polypeptide is a polypeptide comprising a self-cleaving viral peptide and TREX2.

One of the major advantages of the HSV-based vectors contemplated herein, is the ability to transiently regulate the activity of the genome editing platform by flanking the expression cassettes or the polynucleotides therein and/or DNA donor template for altering the genome by one or more guide RNAs, e.g., crRNAs or sgRNAs. When crRNAs are used a corresponding tracRNA is required for each target site for Cas9; tracRNA is not required for cleavage with Cpf1. In one embodiment, the one or more guide RNAs recognize the guide RNA target sites flanking the expression cassettes to inactivate (by excision) the desired components of the genome editing platform and optionally to release the DNA template. In one embodiment, the guide RNA target site flanking the 5′ end of a polynucleotide to be deleted and the guide RNA target site flanking the 3′ of the polynucleotide to be inactivated and optionally to release the DNA template are the same. In one embodiment, the 5′ and 3′ guide RNA target sites flanking the polynucleotide to be inactivated and optionally to release the DNA template are different. In one embodiment, the 5′ and 3′ guide RNA target sites flanking the polynucleotide to be inactivated are the same for all flanked nucleotides and optionally to release the DNA template. In one embodiment, the 5′ and 3′ guide RNA target sites flanking the polynucleotide to be inactivated are different for all flanked nucleotides and optionally to release the DNA template, but in some embodiments, all the 5′ guide RNA target sites are the same and all the 3′ guide RNA target sites are the same.

In various embodiments, the same one or more guide RNAs target both the guide RNA targets sites in the vector as well as the target sequence in the genome. See, e.g., SEQ ID NOs: 1-55.

In particular embodiments, HSV-based vectors comprises one or more expression cassettes comprising a RNA pol II promoter. The promoters may be ubiquitous or constitutive RNA pol II promoters, tissue or lineage-specific RNA pol II promoters, or inducible RNA pol II promoters.

Illustrative examples of ubiquitous RNA pol II promoters useful in certain embodiments contemplated herein include, but are not limited to cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

Illustrative examples of tissue specific or lineage specific RNA pol II promoters useful in certain embodiments contemplated herein include, but are not limited to a B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

Other illustrative examples of tissue specific or lineage specific RNA pol II promoters useful in certain embodiments contemplated herein include, but are not limited to an hSYN1 promoter, a TRPV1 promoter, a Naᵥ1.7 promoter, a Naᵥ1.8 promoter, and a Naᵥ1.9 promoter.

Additional illustrative examples of tissue specific or lineage specific RNA pol II promoters useful in certain embodiments contemplated herein include, but are not limited to a promoter specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), or the like

In particular embodiments, an HSV-based vector comprises a third expression cassette that encodes a switch polypeptide selected from the group consisting of a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

Illustrative examples of inducible RNA pol II promoters useful in certain embodiments contemplated herein include, but are not limited to a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

In particular embodiments, an HSV-based vector comprises one or more expression cassettes comprising a RNA pol III promoter. The promoters may be ubiquitous or constitutive RNA pol III promoters or inducible RNA pol III promoters.

Illustrative examples of ubiquitous RNA pol III promoters useful in certain embodiments contemplated herein include, but are not limited to a human U6 snRNA promoter, a mouse U6 snRNA promoter, a human H1 RNA promoter, a mouse H1 RNA promoter, and a human tRNA-val promoter.

Illustrative examples of inducible RNA pol III promoters useful in certain embodiments contemplated herein include, but are not limited to an RNA pol III promoter operably linked to a tetracycline responsive regulatory element or a peroxide inducible regulatory element.

In particular embodiments, the HSV-based vector contains a first expression cassette that comprises a polynucleotide encoding a CRISPR-Cas endonuclease selected from the group consisting of: Cpf1, Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. In a preferred embodiment, the Cas is Cas9 or Cpf1. In particular embodiments, the Cas9 or Cpf1 may comprise one or more mutations in a HNH or a RuvC-like endonuclease domain or the HNH and the RuvC-like endonuclease domains as disclosed elsewhere herein.

In particular embodiments, an HSV-based vector contemplated herein comprises a polynucleotide encoding a DNA template for altering at least one site in a genome. The alteration may comprise correction of one or more genome sequences or insertion of sequences into the genome.

In some embodiments, the editing of the genome in the cell comprises insertion of a regulatable transcriptional regulatory element upstream of a transcription start site in a gene of the cell.

In certain embodiments, the transcriptional regulatory element may be activated in the presence of an exogenous ligand or small molecule or activated in the absence of an exogenous ligand or small molecule.

In certain other embodiments, the transcriptional regulatory element may be repressed in the presence of an exogenous ligand or small molecule or repressed in the absence of an exogenous ligand or small molecule.

In one embodiment, the r transcriptional regulatory element is inserted upstream of a gene associated with the regulation of pain.

In a particular embodiment, the transcriptional regulatory element is inserted upstream of a gene encoding a voltage gated sodium channel.

Illustrative examples of voltage gated sodium channels include, but are not limited to: Naᵥ1.1, Naᵥ1.3, Naᵥ1.6, Naᵥ1.7, Naᵥ1.8, and Naᵥ1.9.

In one embodiment, a DNA templates contemplated herein, can be designed to correct or alter a polynucleotide sequence involved in the pathogenesis of a monogenetic disease, checkpoint genes in T cells used for T cell therapy, nociceptive genes, genes in viral genomes, genes involved in neurodegenerative diseases, genes involved in polycystic kidney disease, Walker-Warburg syndrome, genes involved in trinucleotide repeat diseases, genes involved in inflammatory disease, genes involved in cancer, genes involved in liver disease, genes involved in retinal diseases, polynucleotide sequences that contribute to aberrant splicing, and interregulatory genes.

One having ordinary skill in the art would recognize that the vector and compositions contemplated herein are not limited by any particular target sequence and that the genome editing platform could be designed to provide knockout/disruption or correction/insertion of any genomic locus where the sequence is known.

In various embodiments, any of the foregoing vector elements may be combined in various combinations and orientations. The skilled artisan would appreciate that many other different embodiments can be fashioned from the existing embodiments of the invention.

A “host cell” includes cells transfected, infected, or transduced in vivo, ex vivo, or in vitro with a recombinant vector or a polynucleotide of the invention. Host cells may include virus producing cells and cells infected with viral vectors. In particular embodiments, host cells infected with viral vector contemplated are administered to a subject in need of therapy. In certain embodiments, the term “target cell” is used interchangeably with host cell and refers to infected cells of a desired cell type.

Replication-deficient HSV-based vectors are typically produced in complementing cell lines that provide gene functions not present in the replication-deficient HSV-based vectors, but required for viral propagation, at appropriate levels in order to generate high titers of viral vector stock. A preferred cell line complements for at least one and preferably all replication-essential gene functions not present in a replication-deficient HSV-based vector. For example, a HSV-based vector deficient in ICP0, ICP4, ICP22, ICP27, and ICP47 is complemented by the human osteosarcoma line U2OS (Yao and Schaffer, 1995). The cell line also can complement non-essential genes that, when missing, reduce growth or replication efficiency (e.g., UL55). The complementing cell line can complement for a deficiency in at least one replication-essential gene function encoded by the early regions, immediate-early regions, late regions, viral packaging regions, virus-associated regions, or combinations thereof, including all HSV functions (e.g., to enable propagation of HSV amplicons, which comprise minimal HSV sequences, such as only inverted terminal repeats and the packaging signal or only ITRs and an HSV promoter). The cell line preferably is further characterized in that it contains the complementing genes in a non-overlapping fashion with the HSV-based vector, which minimizes, and practically eliminates, the possibility of the HSV-based vector genome recombining with the cellular DNA. Accordingly, the presence of replication competent HSV is minimized, if not avoided in the vector stock, which, therefore, is suitable for certain therapeutic purposes, especially gene therapy purposes. The construction of complementing cell lines involves standard molecular biology and cell culture techniques well known in the art.

In some embodiments, the HSV-based vector is deficient in replication-essential gene functions of only the early regions of the HSV genome, only the immediate-early regions of the HSV genome, only the late regions of the HSV genome, or both the early and late regions of the HSV genome. In a particular embodiment, the HSV-based vector also can have essentially the entire HSV genome removed, in which case it is preferred, in certain embodiments, that at least either the viral inverted terminal repeats (ITRs) and one or more promoters or the viral ITRs and a packaging signal are left intact (i.e., an HSV amplicon). The larger the region of the HSV genome that is removed, the larger the piece of exogenous nucleic acid sequence that can be inserted into the genome.

In particular embodiments, one or more envelope proteins of the HSV-based vector can be modified so as to alter the binding specificity, attachment, and/or entry of the HSV-based vector to a particular cell. For targeting HSV-based vectors, such manipulations can include deletion of regions of any one of the ten glycoproteins (gB, gC, gD, gE, gG, gH, gI, gJ, gL, and gM) and four non-glycosylated integral membrane proteins (UL20, UL34, UL45, and UL49.5), or insertions of various native or non-native ligands into portions of an envelope glycoprotein. Manipulation of the envelope proteins can broaden the range of cells infected by the HSV-based vector or enable targeting of the HSV-based vector to a specific cell type.

In particular embodiments, the HSV-based vector can be modified so as to alter the binding specificity, attachment, and/or entry of the HSV-based vector to a particular cell by replacement of a portion of gD (such as of residues 61-218) with an antibody or antigen binding fragment thereof, e.g., scFv targeting a cell surface protein expressed on a target cell.

In particular embodiments, an HSV viral stock is provided, preferably a homogeneous stock, comprising the HSV-based vector. The preparation and analysis of HSV stocks is well known in the art. For example, a viral stock can be manufactured in roller bottles containing cells transduced with the HSV vector. The viral stock can then be purified on a continuous nycodenze gradient, and aliquotted and stored until needed. Viral stocks vary considerably in titer, depending largely on viral genotype and the protocol and cell lines used to prepare them.

In particular embodiments, the titer of an HSV-based vector contemplated herein is at least about 10⁵ plaque-forming units (pfu), such as at least about 10⁶ pfu or even more preferably at least about 10⁷ pfu. In certain embodiments, the titer can be at least about 10⁸ pfu, or at least about 10⁹ pfu, and high titer stocks of at least about 10¹⁰ pfu or at least about 10¹¹ pfu are most preferred. Such titers are established using cells that express the targeted receptor.

### F. Genetically Modified Cells

The present invention contemplates, in particular embodiments, cells genetically modified to express the genome engineering platforms contemplated herein, for use in gene therapy.

As used herein, the term “genetically engineered” or “genetically modified” or “genome editing” refers to the addition, deletion, or modification of the genetic material in a cell using the vectors, compositions, and methods contemplated herein. The terms, “genetically modified cells,” “modified cells,” and, “redirected cells,” are used interchangeably. In particular embodiments, cells infected with vectors contemplated herein are genetically modified once the components of the genome editing platform are produced in the cell. It is contemplated that the improved HSV-based vectors and genome editing platforms disclosed herein will produce genetically modified cells with fewer off-target effects than existing methods of gene therapy.

As used herein, the term “gene therapy” refers to the introduction of extra genetic material in the form of DNA or RNA into the total genetic material in a cell that restores, corrects, or modifies the cell's physiology to provide a desired therapeutic outcome.

In various embodiments, the genetically modified cells contemplated herein are infected in vivo, in vitro or ex vivo with vectors contemplated herein. Cells genetically modified ex vivo may optionally be expanded ex vivo prior to use in vivo. The genetically modified cells are then administered to a subject in need of gene therapy.

Illustrative examples of cells suitable for use in the genome editing and gene therapy methods contemplated herein include, but are not limited to stem cells, progenitor cells, and differentiated cells.

In particular embodiments, cells suitable for use in the genome editing and gene therapy methods contemplated herein include, but are not limited to mesenchymal stem and/or progenitor cells, mesodermal stem and/or progenitor cells, endodermal stem and/or progenitor cells, or ectodermal stem and/or progenitor cells.

In certain embodiments, cells suitable for use in the genome editing and gene therapy methods contemplated herein include, but are not limited to bone marrow stem cells, umbilical cord blood stem and/or progenitor cells, bone stem and/or progenitor cells, muscle stem and/or progenitor cells, hematopoietic stem and/or progenitor cells, fat stem and/or progenitor cells, cartilage stem and/or progenitor cells, neural stem and/or progenitor cells, skin stem and/or progenitor cells, liver stem and/or progenitor cells, pancreas stem and/or progenitor cells, kidney stem and/or progenitor cells, gastric stem and/or progenitor cells, and intestinal stem and/or progenitor cells.

In certain embodiments, cells suitable for use in the genome editing and gene therapy methods contemplated herein include, but are not limited to embryonic stem cells, bone marrow stem cells, umbilical cord stem cells, placental stem cells, mesenchymal stem cells, hematopoietic stem cells, erythroid progenitor cells, and erythroid cells.

In particular embodiments, cells suitable for use in the genome editing and gene therapy methods contemplated herein include, but are not limited to pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like.

In one embodiment, the cells are neural cells. Illustrative examples of neural cells include, but are not limited to sensory neurons, nociceptive neurons, dorsal root ganglion neurons, C-fibers, αδ-fibers, and neurons that express aTRP receptor, e.g., TRPV1, TRPV2.

In one embodiment, the cells are hematopoietic cells. Illustrative examples of hematopoietic cells include HSCs, HPCs, T cells, CD34+ cells, and the like. Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

In particular embodiments, hematopoietic stem cells are isolated from bone marrow, umbilical cord blood, or peripheral circulation.

In one embodiment, the hematopoietic cells are HSCs or HPCs. In one embodiment, the hematopoietic cells are CD34⁺ cells.

Cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic. An “isolated cell” refers to a cell that has been obtained from an in vivo tissue or organ and is substantially free of extracellular matrix.

### G. Compositions and Formulations

The present invention further includes various pharmaceutical compositions comprising polynucleotides, vectors, polypeptides, or genetically modified cells produced according to methods described herein and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the invention is contemplated.

The compositions of the invention may comprise one or more polypeptides, polynucleotides, vectors comprising same, infected cells, etc., as described herein, formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules or various pharmaceutically-active agents. There is virtually no limit to other components that may also be included in the compositions, provided that the additional agents do not adversely affect the ability of the composition to deliver the intended gene therapy.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain circumstances it will be desirable to deliver the compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabenes, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, optionally mixing with CPP polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the invention may comprise one or more polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the invention may be administered in combination with other agents as well, such as, e.g., cells, other proteins or polypeptides or various pharmaceutically-active agents.

In a particular embodiment, a formulation or composition according to the present invention comprises a cell contacted with a combination of any number of polypeptides, polynucleotides, and viral vectors, as contemplated herein.

In certain aspects, the present invention provides formulations or compositions suitable for the delivery of viral vector systems.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain aspects, the present invention provides pharmaceutically acceptable compositions which comprise a therapeutically-effective amount of one or more polynucleotides or polypeptides, as described herein, formulated together with one or more pharmaceutically acceptable carriers (additives) and/or diluents (e.g., pharmaceutically acceptable cell culture medium).

Particular embodiments of the invention may comprise other formulations, such as those that are well known in the pharmaceutical art, and are described, for example, in Remington: The Science and Practice of Pharmacy, 20th Edition. Baltimore, Md.: Lippincott Williams & Wilkins, 2000.

### H. Genome Editing Methods

In various embodiments, an HSV-based vector contemplated herein provides curative, preventative, or ameliorative benefits to a subject diagnosed with or that is suspected of having monogenic disease, disorder, or condition or a disease, disorder, or condition amenable to genome editing. The virus can infect the cell in vivo, ex vivo, or in vitro. In ex vivo and in vitro embodiments, the infected cells can then be administered to a subject in need of therapy. In various embodiments, vectors, viral particles, and genetically modified cells of the invention are be used to treat, prevent, and/or ameliorate a monogenic disease, disorder, or condition or a disease, disorder, or condition amenable to genome editing in a subject. In various embodiments, the viral vectors are administered by direct injection to a cell, tissue, or organ of a subject in need of gene therapy, in vivo. In various other embodiments, cells are infected and optionally expanded in vitro or ex vivo with vectors contemplated herein. The infected cells are then administered to a subject in need of therapy.

A “subject,” as used herein, includes any animal that exhibits a symptom of a monogenic disease, disorder, or condition that can be treated with the HSV-based vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by genome editing.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In various embodiments, a subject in need of a cell-based therapy is administered a population of cells comprising an effective amount of genetically modified cells contemplated herein.

As used herein, the term “amount” refers to “an amount effective” or “an effective amount” of a virus or genetically modified therapeutic cell to achieve a beneficial or desired prophylactic or therapeutic result, including clinical results.

A “prophylactically effective amount” refers to an amount of a virus or genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient).

In one embodiment, the present invention provides a method of providing genetically modified cell to a subject that comprises administering, e.g., parenterally, one or more cells transduced with a vector contemplated herein.

In various embodiments, HSV-based vectors contemplated herein can be used to knockout or disrupt a gene or genetic regulatory sequence, correct a sequence in the genome, or insert genetic material into the genome. The HSV-based vectors comprise one or more guide RNAs that function to target the Cas endonuclease to one ore more target sites to facilitate altering the genome.

Illustrative examples of target sites include a sequence associated with a signaling biochemical pathway, e.g., a signaling biochemical pathway-associated gene or polynucleotide. Further illustrative examples of target sites also include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

In one embodiment, a method of genetically modifying a cell comprises introducing an HSV-based vector contemplated herein into the cell and inducing the expression of the switch polypeptide for a time sufficient to edit the genome of the cell. In a particular embodiment, a nociceptive gene is disrupted thereby enabling the treatment, prevent, or amelioration of pain. In one embodiment, the nociceptive gene is a voltage gated sodium channel. In a preferred embodiment the voltage gated sodium channel is selected from the group consisting of: Naᵥ1.1, Naᵥ1.3, Naᵥ1.6, Naᵥ1.7, Naᵥ1.8, and Naᵥ1.9.

In a particular embodiment, the editing of the genome in the cell comprises insertion of a regulatable transcriptional regulatory element upstream of a transcription start site in a gene of the cell. The transcriptional regulatory element can be activated or repressed in the presence or absence of an exogenous ligand or small molecule. In a particular embodiment, the regulatable transcriptional regulatory element is inserted upstream of a nociceptive gene thereby enabling the transcriptional control of the gene and facilitating the treatment, prevent, or amelioration of pain. In one embodiment, the nociceptive gene is a voltage gated sodium channel. In a preferred embodiment the voltage gated sodium channel is selected from the group consisting of: Naᵥ1.1, Naᵥ1.3, Naᵥ1.6, Naᵥ1.7, Naᵥ1.8, and Naᵥ1.9.

Examples of disease-associated genes and polynucleotides as well as examples of genes associated with biochemical signaling pathways are disclosed in U.S. Pat. No. 8,945,839, Tables A, B, and C, each of which is included herein by reference in its entirety.

In addition, the present invention contemplates identifying one or more guide RNA target sites for gene editing in any of the 9578 disease associated genes disclosed, along with the corresponding diseases, in GeneCards database as of Oct. 27, 2015 (www.genecards.org/cgi-bin/listdiseasecards.pl?type=full), the disclosure of which is incorporated herein in its entirety.

In various embodiments, one or more crRNAs or sgRNAs contemplated herein, can be designed to target a polynucleotide sequence involved in the pathogenesis of a monogenetic disease, to modify checkpoint genes in T cells used for T cell therapy, to modify nociceptive genes, to modify genes in viral genomes, to modify genes involved in neurodegenerative diseases, to modify genes involved in polycystic kidney disease, Walker-Warburg syndrome, to modify genes involved in trinucleotide repeat diseases, to modify genes involved in inflammatory disease, to modify genes involved in cancer, to modify genes involved in liver disease, to modify genes involved in retinal diseases, polynucleotide sequences that contribute to aberrant splicing, and to modify genes interregulatory genes.

In one embodiment, the HSV-based vectors contemplated herein can be used to knockout or disrupt a gene or genetic regulatory sequence encoding a checkpoint gene in a T cell so as to make the T cell “invisible” to checkpoint gene products and increase the therapeutic efficacy of the T cell.

In a particular embodiment, the HSV-based vectors contemplated herein can be used to knockout or disrupt a gene or genetic regulatory sequence, correct a sequence in the genome, or insert genetic material into the genome to treat a hemoglobinopathy in a subject. A “hemoglobinopathy” or “hemoglobinopathic condition” includes any disorder involving the presence of an abnormal hemoglobin molecule in the blood. Examples of hemoglobinopathies included, but are not limited to, hemoglobin sickle cell disease (SCD), hemoglobin S (HbS) disease, hemoglobin C (HbC) disease, hemoglobin D (HbD) disease, hemoglobin E (HbE) disease, and thalassemias. Also included are hemoglobinopathies in which a combination of abnormal hemoglobins are present in the blood, e.g., HbS/beta thalassemia disease (Sβ⁺ or Sβ⁰); HbS/HbC; HbS/HbD; and HbS/HbE. In a particular embodiment, the hemoglobinopathy is selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease.

In a particular embodiment, the HSV-based vectors contemplated herein can be used to knockout or disrupt a nociceptive gene to treat neuropathic pain. Illustrative examples of nociceptive genes include, but are not limited to CSF-1, Naᵥ1.7, Naᵥ 1.8, and Naᵥ1.9.

In another particular embodiment, the HSV-based vectors contemplated herein can be used to knockout or disrupt a viral gene for the destruction of latent viral genomes, e.g., from HIV, HSV, or HPV.

In one embodiment, the HSV-based vectors contemplated herein can be used to knockout or disrupt a region of trinucleotide repeat expansion in a gene involved in neurodegenerative diseases, including but not limited to HTT, ATN1 or DRPLA, AR, ATXN1, ATXN2, ATXN3, CACNA1A, ATXN7, TBP, FMR1, AFF2, FMR2, FXN, DMPK, OSCA, SCAB, PPP2R2B, OR SCA12.

In one embodiment, the HSV-based vectors contemplated herein can be used to knockout or disrupt a region involved in polycystic kidney disease or Walker-Warburg syndrome.

In one embodiment, the HSV-based vectors contemplated herein can be used to knockout or disrupt CSF-1 production to deplete microglia and treat neuroinflammatory diseases such as Alzheimer's disease and Parkinson's disease.

In one embodiment, the HSV-based vectors contemplated herein can be used to correct a sequence in FVIII or insert a copy of a normal copy of the FVIII gene in a cell in order to treat hemophilia.

In another embodiment, the HSV-based vectors contemplated herein can be used to correct a sequence in the genome, or insert genetic material into the genome to treat a retinal disease, e.g, correct a rhodopsin mutation to treat retinitis pigmentosa or correct a mutation in ABCA4 to treat Stargardt disease.

In one embodiment, the HSV-based vectors contemplated herein can be used to correct a sequence in the genome, or insert genetic material into the genome to treat a disease resulting from aberrant splicing, e.g., Familial dysautonomia, Frontotemporal lobar dementias/-amyotrophic lateral sclerosis, Hutchinson-Gilford progeria syndrome, Medium-chain acyl-CoA dehydrogenase (MCAD) deficiency, Myotonic dystrophy, Prader-Willi syndrome, Spinal muscular atrophy, Tauopathies, as well as correcting mutations in tumor suppressor genes, including APC, BRCA1, Estrogen receptor, NF1, NF2, and MLH1.

In a particular embodiment, the HSV-based vectors contemplated herein can be used to knockout or disrupt a gene or genetic regulatory sequence, correct a sequence in the genome, or insert genetic material into the genome to alter an interregulatory gene.

Further illustrative examples of conditions treatable with the genome editing platforms contemplated herein include: Aicardi-Goutières Syndrome; Alexander Disease; Allan-Herndon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum.

Viral vectors of the present invention may be used to alter the expression of one or more mammalian polypeptides useful in the treatment of a disease, disorder, or condition that is amenable to treatment with gene therapy, including, but not limited to monogenic diseases, disorders, and conditions. The vectors may be used to correct a defective copy of a gene involved in a monogenetic disease or provide one or more additional healthy copies of a gene involved in a monogenetic disease.

1. Monogenic Diseases

As used herein, the term “monogenic disease” refers to a disease in which modifications of a single gene is associated with a disorder, disease, or condition in a subject. Though relatively rare, monogenic diseases affect millions of people worldwide. Scientists currently estimate that over 10,000 human diseases are known to be monogenic. Pure genetic diseases are caused by a single error in a single gene in the human DNA. The nature of disease depends on the functions performed by the modified gene. The single-gene or monogenic diseases can be classified into three main categories: Dominant, Recessive, and X-linked. All human beings have two sets or copies of each gene called “allele”; one copy on each side of the chromosome pair. Recessive diseases are monogenic disorders that occur due to damages in both copies or alleles. Dominant diseases are monogenic disorders that involve damage to only one gene copy. X linked diseases are monogenic disorders that are linked to defective genes on the X chromosome which is the sex chromosome. The X linked alleles can also be dominant or recessive. These alleles are expressed equally in men and women, more so in men as they carry only one copy of X chromosome (XY) whereas women carry two (XX).

Monogenic diseases are responsible for a heavy loss of life. The global prevalence of all single gene diseases at birth is approximately 1/100. In Canada, it has been estimated that taken together, monogenic diseases may account for up to 40% of the work of hospital based pediatric practice (Scriver, 1995).

Illustrative embodiments of monogenic disorders, diseases, and conditions that can be treated, prevented, or ameliorated with compositions and methods contemplated herein include, but are not limited to: 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis; anauxetic dysplasia; androgen insentivity syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT1 deficiency; glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborm errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome Imag; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1 Imag; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann corneal dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

Other illustrative embodiments of diseases, disorders, and conditions that can be treated, prevented, or ameliorated using methods and compositions of the present invention include, but not limited to treating macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The genetically modified cells may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment, a dose of genetically modified cells is delivered to a subject intravenously. In preferred embodiments, genetically modified cells are intravenously administered to a subject.

In particular embodiments, patients receive a dose of genetically modified cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In certain embodiments, patients receive a dose of genetically modified cells of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of genetically modified cells of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods of the invention provide more robust and safe gene therapy than existing methods and comprise administering a population or dose of cells comprising about 5% genetically modified cells, about 10% genetically modified cells, about 15% genetically modified cells, about 20% genetically modified cells, about 25% genetically modified cells, about 30% genetically modified cells, about 35% genetically modified cells, about 40% genetically modified cells, about 45% genetically modified cells, about 50% genetically modified cells, about 55% genetically modified cells about 60% genetically modified cells about 65% genetically modified cells about 70% genetically modified cells about 75% genetically modified cells about 80% genetically modified cells about 85% genetically modified cells about 90% genetically modified cells about 95% genetically modified cells, or about 100% genetically modified cells to a subject.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

TABLE 3
SEQ ID NOs: 1-55

| SEQ | |
|---|---|
| ID | |
| NO | SEQUENCE (5′ to 3′) |
| 1 | GTAGCAGCCCCGCCTCCCCGAGG |
| 2 | GGGCGCAGTCTGCTTGCAGGCGG |
| 3 | TGGGCTAGCCTGGGTGCCAGTGG |
| 4 | TGGGTGCCAGTGGCTGCTAGCGG |
| 5 | GCAGCCCTGAGAGAGCGCCGGGG |
| 6 | GCAGAATCTGGCTCCAGGAGAGG |
| 7 | GACAAAGCTCTGAGGTCCTGGGG |
| 8 | GACCAAAAATGTCGAGTAAGTGG |
| 9 | AGAGAGCGCCGGGGAAGGAGAGG |
| 10 | GTGAAATGGACAAAGCTCTGAGG |
| 11 | GGCTCTGACACCATGCCGGGAGG |
| 12 | AGGCGGGGCTGCTACCTCCACGG |
| 13 | GGCGGGGCTGCTACCTCCACGGG |
| 14 | AGACTGCGCCCCTCCTGCCAGGG |
| 15 | GAGGGGCGCAGTCTGCTTGCAGG |
| 16 | AGCGCTCCAGCGGCGGCTGTCGG |
| 17 | GGAAAGCCGACAGCCGCCGCTGG |
| 18 | GCTTTCCAATTCCGCCAGCTCGG |
| 19 | GCTAGCAGCCACTGGCACCCAGG |
| 20 | TGCCAGTGGCTGCTAGCGGCAGG |
| 21 | CGCCTGCCGCTAGCAGCCACTGG |
| 22 | GCAGGCGTCCCCTGAGCAACAGG |
| 23 | AGGGCTGCTTCTTTTTCTCTGGG |
| 24 | AGCAGCCCTGAGAGAGCGCCGGG |
| 25 | GATTCTGCAGGTGCACTGGGTGG |
| 26 | GTGCACTGGGTGGGGATGATCGG |
| 27 | GTGGGGATGATCGGCGGGCTAGG |
| 28 | GGACAAAGCTCTGAGGTCCTGGG |
| 29 | TGGACAAAGCTCTGAGGTCCTGG |
| 30 | AGCAATGCGTTGTTCAATGAGGG |
| 31 | GGAATGTCCCCATAGATGAAGGG |
| 32 | AGGAATGTCCCCATAGATGAAGG |
| 33 | TGGGGACATTCCTCCCGGCATGG |
| 34 | CGGCATGGTGTCAGAGCCCCTGG |
| 35 | GACAGCCGCCGCTGGAGCGCTGG |
| 36 | GAGAGGCCCGCGCCCTCTCCTGG |
| 37 | GCCAGATTCTGCAGGTGCACTGG |
| 38 | AGGGGCTCTGACACCATGCCGGG |
| 39 | CGGCTGAGGCTGGGCTAGCCTGG |
| 40 | AGCTCCTCACATAAGAGGCCTGG |
| 41 | GTCTGCAGGAGGAAAAAGAAAGG |
| 42 | GTAGGGGTCCAAGTCCTCCAGGG |
| 43 | AGTAGGGGTCCAAGTCCTCCAGG |
| 44 | GTCAGAGCCCCTGGAGGACTTGG |
| 45 | GGCTGAGGCTGGGCTAGCCTGGG |
| 46 | CGGCGCAGCTGCCCTCGGGGAGG |
| 47 | CGCAGCTGCCCTCGGGGAGGCGG |
| 48 | CGGGCGCGCCCTGGCAGGAGGGG |
| 49 | GGCGGTCGCCAGCGCTCCAGCGG |
| 50 | GGTCGCCAGCGCTCCAGCGGCGG |
| 51 | GGCGCGGGCCTCTCCTTCCCCGG |
| 52 | GGAGAAAGCATATATAAAGCAGG |
| 53 | GTGTTTAGGTACACTTTTACTGG |
| 54 | GCAGCTGCCCTCGGGGAGGCGGG |
| 55 | GAAGAGGAATTAAAATATACAGG |

### EXAMPLES

### Example 1

### Construction of an HSV Vector for a Gene Knockout Strategy

An HSV vector was designed as shown in FIG. 1. To amplify the vector, the vector is introduced into the viral producing cell line U2OS-4/27 in the absence of doxycycline. To use the vector for genome editing, the vector is introduced into target cells. In the absence of doxycycline, the rtTA and the gRNAs are expressed. Once the cells are exposed to doxycycline, Cas9 is expressed. Expression of the Cas9 in the presence of the gRNAs generates indels in the gene of interest, and deletes Cas9 and rtTA from the vector.

### Example 2

### Construction of an HSV Vector for a Gene Knockout Strategy

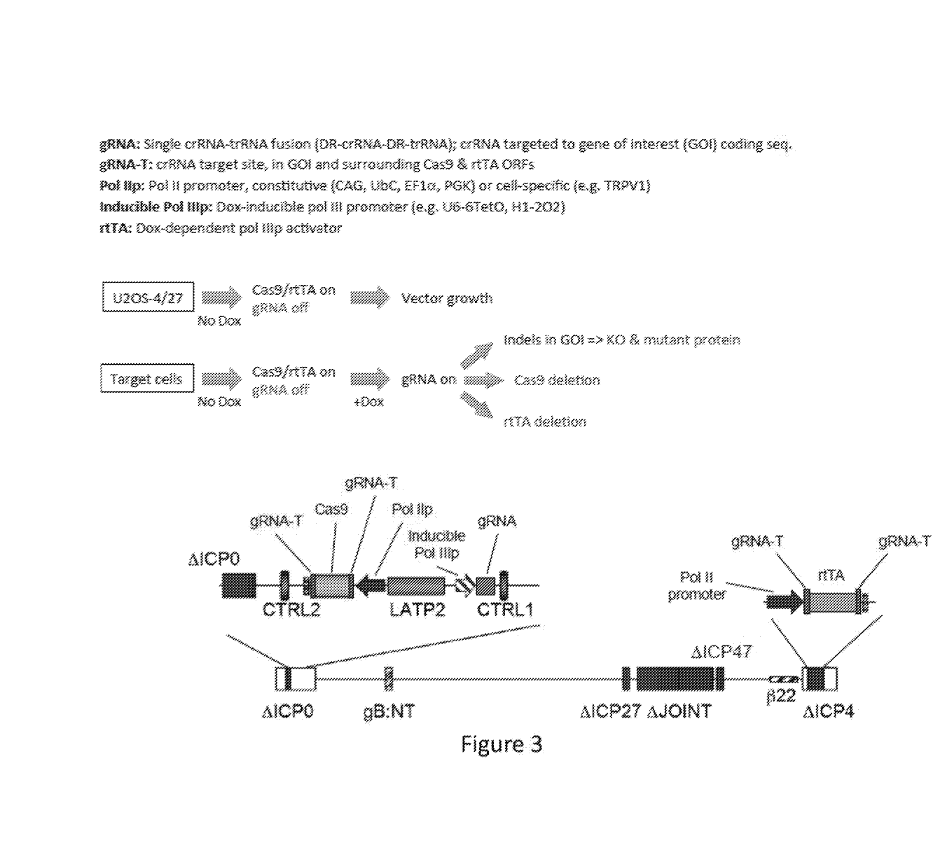

An HSV vector was designed as shown in FIG. 3. To amplify the vector, the vector is introduced into the viral producing cell line U2OS-4/27 in the absence of doxycycline. To use the vector for genome editing, the vector is introduced into target cells. In the absence of doxycycline, the rtTA and the Cas9 are expressed. Once the cells are exposed to doxycycline, the gRNAs are expressed. Expression of the Cas9 in the presence of the gRNAs generates indels in the gene of interest, and deletes Cas9 and rtTA from the vector.

### Example 3

### Construction of an HSV Vector for a

### Cre-Controlled Gene Knockout Strategy

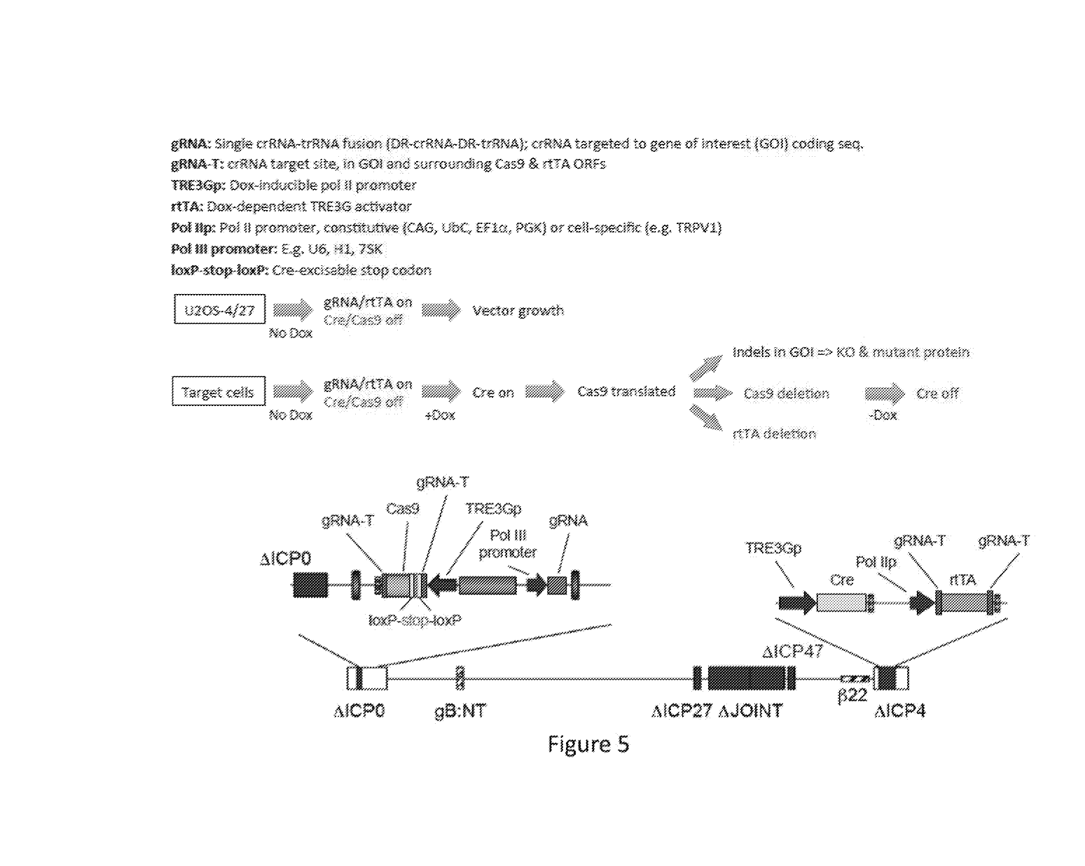

An HSV vector was designed as shown in FIG. 5. To amplify the vector, the vector is introduced into the viral producing cell line U2OS-4/27 in the absence of doxycycline. To use the vector for genome editing, the vector is introduced into target cells. In the absence of doxycycline, the rtTA and the sgRNAs are expressed. Once the cells are exposed to doxycycline, the Cre is expressed and removes the LoxP-stop-LoxP element allowing for transcription and translation of Cas9. Expression of the Cas9 in the presence of the gRNAs generates indels in the gene of interest, and deletes Cas9 and rtTA from the vector. Subsequent removal of doxycycline turns off Cre expression.

### Example 4

### Construction of an HSV Vector for a

### miRNA-Controlled Gene Knockout Strategy

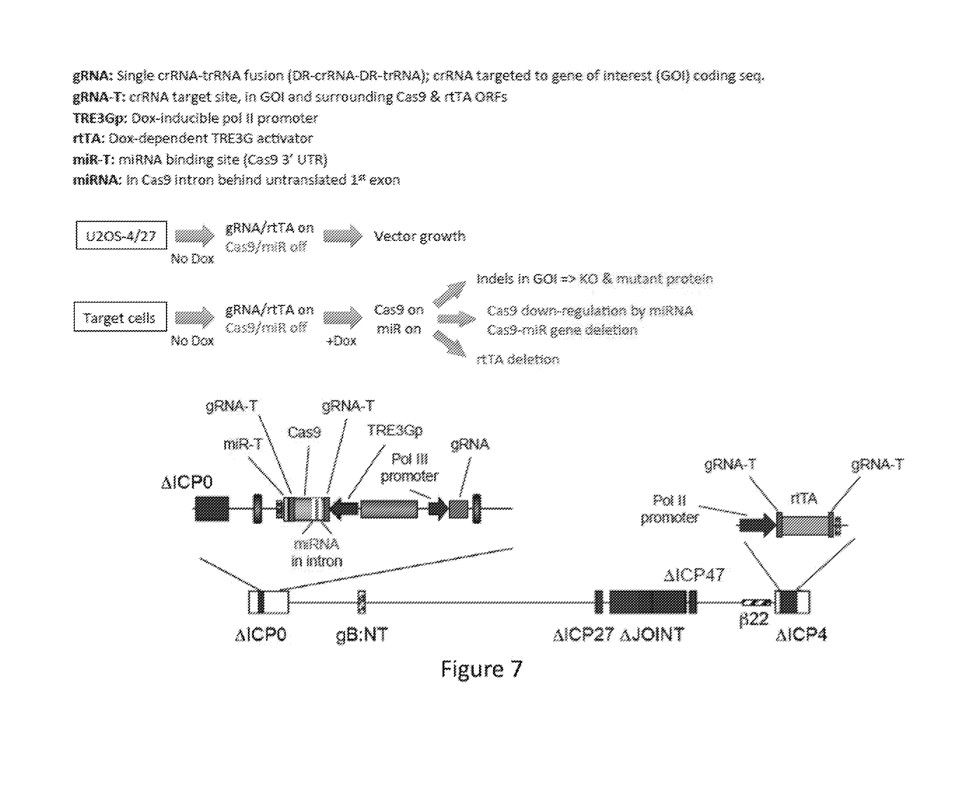

An HSV vector was designed as shown in FIG. 7. To amplify the vector, the vector is introduced into the viral producing cell line U2OS-4/27 in the absence of doxycycline. To use the vector for genome editing, the vector is introduced into target cells. In the absence of doxycycline, the rtTA and the sgRNAs are expressed. Once the cells are exposed to doxycycline, Cas9 is expressed and the miRNA is processed. Expression of the Cas9 in the presence of the gRNAs generates indels in the gene of interest, and deletes Cas9 and rtTA from the vector. An additional layer of safety is afforded in this vector design because the processed mature miRNA then binds to the miRNA site in the Cas9 mRNA, thereby downregulating its expression.

### Example 5

### Construction of an HSV Vector for Gene Correction Strategy

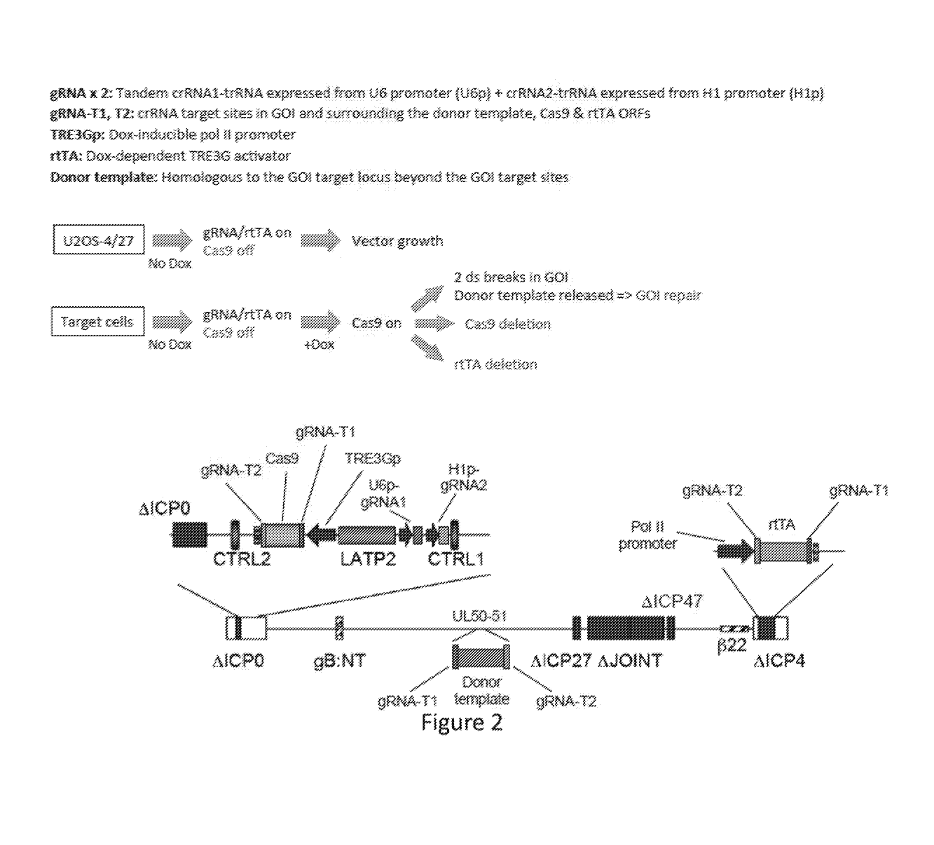

An HSV vector was designed as shown in FIG. 2. To amplify the vector, the vector is introduced into the viral producing cell line U2OS-4/27 in the absence of doxycycline. To use the vector for genome editing, the vector is introduced into target cells. In the absence of doxycycline, the rtTA and the sgRNAs are expressed. Once the cells are exposed to doxycycline, Cas9 is expressed. Expression of the Cas9 in the presence of the gRNAs promotes gene correction by releasing the donor template and making a pair of double strand breaks in the gene of interest. Expression of the Cas9 in the presence of the gRNAs also deletes Cas9 and rtTA from the vector.

Although FIG. 2 shows that the location of the donor template is between UL50 and UL51, the donor template may be inserted into, replace, or flank any of the following loci: ICP0, ICP4, ICP22, ICP27, ICP47, UL2, UL3, UL4, UL10, UL11, UL13, UL16, UL20, UL21, UL23, UL24, UL39, UL40, UL41, UL43, UL44, UL45, UL46, UL47, UL50, UL51, UL55, UL56, LAT, ICP34.5, US1, US2, US3, US4, US5, US7, US8, US9, US10, and US11

### Example 6

### Construction of an HSV Vector for Gene Correction Strategy

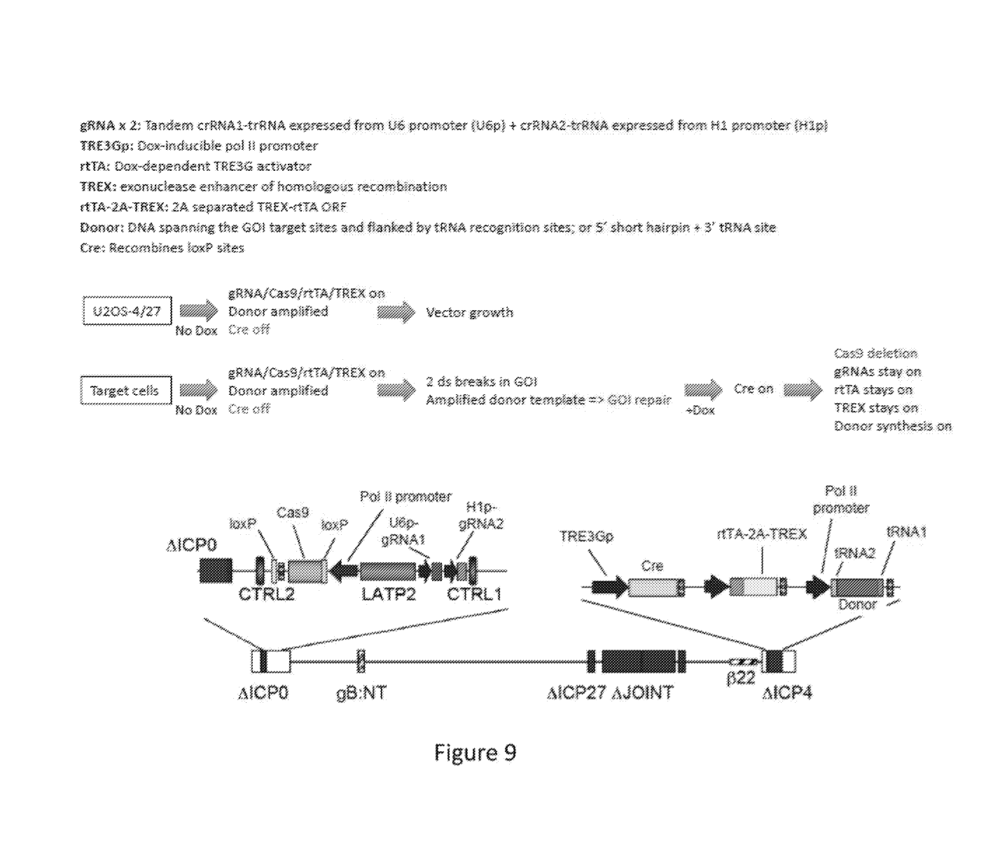

An HSV vector was designed as shown in FIG. 9. To amplify the vector, the vector is introduced into the viral producing cell line U2OS-4/27 in the absence of doxycycline. To use the vector for genome editing, the vector is introduced into target cells. In the absence of doxycycline, Cas9, the rtTA, TREX2, and the sgRNAs are expressed and expression of the Cas9 in the presence of the gRNAs promotes gene correction by releasing the donor template and making a pair of double strand breaks in the gene of interest. TREX2 facilitates the gene correction. Once the cells are exposed to doxycycline, Cre is expressed and Cas9 is deleted from the vector by Cre-mediated recombination between the LoxP sites flanking the polynucleotide encoding Cas9.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An HSV-based vector comprising a CRISPR-Cas system for the treatment, prevention, or amelioration of chronic pain.

2. The HSV-based vector of claim 1, comprising
a) a first expression cassette that comprises an RNA polymerase II promoter operably linked to a polynucleotide encoding a CRISPR-Cas endonuclease; and
b) a second expression cassette that comprises one or more RNA polymerase III promoters operably linked to one or more guide RNAs.

3. An HSV-based vector comprising an inducibly and transiently regulatable CRISPR-Cas system.

4. The HSV-based vector of claim 3, comprising
a) a first expression cassette that comprises at least one regulatory element for inducible expression and at least one regulatory element for transient expression and a polynucleotide encoding a CRISPR-Cas endonuclease;
b) a second expression cassette that comprises a polynucleotide encoding one or more guide RNAs; and
c) a third expression cassette that comprises a polynucleotide encoding a switch polypeptide that binds to the at least one element for inducible expression.

5. The HSV-based vector of claim 3, comprising
a) a first expression cassette that comprises at least one regulatory element for transient expression and a polynucleotide encoding a CRISPR-Cas endonuclease;
b) a second expression cassette that comprises at least one regulatory element for inducible expression and a polynucleotide encoding one or more guide RNAs; and
c) a third expression cassette that comprises a polynucleotide encoding a switch polypeptide that binds to the at least one element for inducible expression; and optionally, at least one regulatory element for transient expression.

6. The HSV-based vector of any one of preceding claims, wherein the one or more guide RNAs target one or more guide RNA target sites in the genome.

7. The HSV-based vector of any one of the preceding claims, wherein the at least one regulatory element for transient expression comprises one or more guide RNA target sites.

8. The HSV-based vector of any one of the preceding claims, wherein the at least one regulatory element for transient expression comprises one or more guide RNA target sites and wherein the polynucleotide encoding the CRISPR-Cas endonuclease is flanked by the one or more guide RNA target sites.

9. The HSV-based vector of any one of the preceding claims, wherein the at least one regulatory element for transient expression comprises one or more guide RNA target sites and wherein the polynucleotide encoding the switch polypeptide is flanked by one or more guide RNA target sites.

10. The HSV-based vector of any one of the preceding claims, further comprising a polynucleotide encoding a template for altering at least one site in a genome that is flanked by one or more guide RNA target sites.

11. The HSV-based vector of any one of claims 7-10, wherein the guide RNA target sites flanking any one of the polynucleotide encoding the CRISPR-Cas endonuclease, the polynucleotide encoding the switch polypeptide, and the polynucleotide encoding the template for altering at least one site in the genome are the same.

12. The HSV-based vector of any one of claims 7-10, wherein the guide RNA target sites flanking the polynucleotide encoding the CRISPR-Cas endonuclease, the polynucleotide encoding the switch polypeptide, and the polynucleotide encoding the template for altering at least one site in the genome are the same.

13. The HSV-based vector of any one of claims 7-10, wherein each of the guide RNA target sites flanking the 5′ end of the polynucleotide encoding the CRISPR-Cas endonuclease, the polynucleotide encoding the switch polypeptide, and the polynucleotide encoding the template for altering at least one site in the genome are the same.

14. The HSV-based vector of any one of claims 7-10, wherein each of the guide RNA target site flanking the 3′ end of the polynucleotide encoding the CRISPR-Cas endonuclease, the polynucleotide encoding the switch polypeptide, and the polynucleotide encoding the template for altering at least one site in the genome are the same.

15. The HSV-based vector of claim 13 or 14, wherein the guide RNA target site flanking the 5′ end and the guide RNA target site flanking the 3′ end of any one of the polynucleotides encoding the CRISPR-Cas endonuclease, the switch polypeptide, and the template for altering at least one site in the genome are different.

16. The HSV-based vector of claim 13 or 14, wherein the guide RNA target site flanking the 5′ end and the guide RNA target site flanking the 3′ end of the polynucleotides encoding the CRISPR-Cas endonuclease, the switch polypeptide, and the template for altering at least one site in the genome are different.

17. The HSV-based vector of any one of claims 13-16, wherein each of the guide RNAs target sites flanking the 5′ end of the polynucleotides encoding the CRISPR-Cas endonuclease, the switch polypeptide, and the template for altering at least one site in the genome are the same; wherein each of the guide RNAs target sites flanking the 3′ end of the polynucleotides encoding the CRISPR-Cas endonuclease, the switch polypeptide, and the template for altering at least one site in the genome are the same; and wherein the guide RNA target site flanking the 5′ end each polynucleotide is different from the guide RNA target site flanking the 3′ end of each of polynucleotide.

18. The HSV-based vector of any one of claims 6-17, wherein the one or more guide RNA target sites in the HSV-based vector are identical to one or more guide RNA target sites in the genome.

19. The HSV-based vector of any one of claims 6-17, wherein the guide RNA target site flanking the 5′ end of each polynucleotide is identical to a guide RNA target site in the genome; wherein the guide RNA target site flanking the 3′ end of each polynucleotide is identical to a guide RNA target site in the genome; and wherein the guide RNA target site flanking the 5′ end each polynucleotide is different from the guide RNA target site flanking the 3′ end of each of polynucleotide.

20. The HSV-based vector of any one of claims 1-19, wherein the second expression cassette comprises one or more guide RNAs that recognize and bind to each of the one or more guide RNAs target sites of any one of claims 6-19.

21. The HSV-based vector of claim 20, wherein the second expression cassette comprises a single guide RNA that recognizes and binds all of the one or more guide RNA target sites of any one of claims 6-19.

22. The HSV-based vector of claim 20, wherein the second expression cassette comprises a plurality of guide RNAs, wherein each of the plurality of guide RNAs recognizes and binds to one of the one or more guide RNA target sites of any one of claims 4-14.

23. The HSV-based vector of any one of the preceding claims, wherein the first expression cassette comprises an HSV promoter, optionally wherein the HSV promoter is the LATP2 promoter.

24. The HSV-based vector of any one of the preceding claims, wherein the first expression cassette comprises an HSV promoter and an RNA polymerase II promoter operably linked to the polynucleotide encoding the CRISPR-Cas endonuclease.

25. The HSV-based vector of claim 24, wherein the RNA polymerase II promoter is a ubiquitous promoter.

26. The HSV-based vector of claim 25, wherein the ubiquitous promoter is selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-MN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

27. The HSV-based vector of claim 24, wherein the RNA polymerase II promoter is a tissue-specific or lineage-specific promoter.

28. The HSV-based vector of claim 27, wherein the tissue-specific or lineage-specific promoter is selected from the group consisting of: a B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

29. The HSV-based vector of claim 27, wherein the tissue-specific or lineage-specific promoter is selected from the group consisting of: an hSYN1 promoter, a TRPV1 promoter, a N₄1.7 promoter, a Naᵥ1.8 promoter, and a N₄1.9 promoter.

30. The HSV-based vector of claim 27, wherein the tissue-specific or lineage-specific promoter is a promoter specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), or the like

31. The HSV-based vector of any one of the preceding claims, wherein the first expression cassette comprises an HSV promoter and/or at least one regulatory element for inducible expression that is operably linked to the polynucleotide encoding the CRISPR-Cas endonuclease.

32. The HSV-based vector of claim 31, wherein at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

33. The HSV-based vector of any one of the preceding claims, wherein the first expression cassette comprises a polynucleotide encoding a CRISPR-Cas endonuclease selected from the group consisting of: Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

34. The HSV-based vector of any one of the preceding claims, wherein the first expression cassette comprises a polynucleotide encoding a Cas9 polypeptide.

35. The HSV-based vector of any one of the preceding claims, wherein the first expression cassette comprises a polynucleotide encoding a Cas9 polypeptide isolated from Staphylococcus aureus, Streptococcus pyogenes, Streptococcus thermophilis, Treponema denticola, and Neisseria meningitidis.

36. The HSV-based vector of any one of claims 33-35, wherein the Cas9 polypeptide comprises one or more mutations in a HNH or a RuvC-like endonuclease domain or the HNH and the RuvC-like endonuclease domains.

37. The HSV-based vector of claim 36, wherein the mutant Cas9 polypeptide is a nickase.

38. The HSV-based vector of claim 36, wherein the mutant Cas9 polypeptide sequence is from Streptococcus pyogenes and comprises a mutation in the RuvC domain.

39. The HSV-based vector of claim 38, wherein the mutation is a D10A mutation.

40. The HSV-based vector of claim 36, wherein the mutant Cas9 polypeptide sequence is from Streptococcus pyogenes and comprises a mutation in the HNH domain.

41. The HSV-based vector of claim 40, wherein the mutation is a D839A, H840A, or N863A mutation.

42. The HSV-based vector of claim 36, wherein the mutant Cas9 polypeptide sequence is from Streptococcus thermophilis and comprises a mutation in the RuvC-like domain.

43. The HSV-based vector of claim 42, wherein the mutation is a D9A mutation.

44. The HSV-based vector of claim 36, wherein the mutant Cas9 polypeptide sequence is from Streptococcus thermophihs and comprises a mutation in the HNH domain.

45. The HSV-based vector of claim 44, wherein the mutation is a D598A, H599A, or N622A mutation.

46. The HSV-based vector of claim 36, wherein the mutant Cas9 polypeptide sequence is from Treponema denticola and comprises a mutation in the RuvC-like domain.

47. The HSV-based vector of claim 46, wherein the mutation is a D13A mutation.

48. The HSV-based vector of claim 36, wherein the mutant Cas9 polypeptide sequence is from Treponema denticola and comprises a mutation in the HNH domain.

49. The HSV-based vector of claim 48, wherein the mutation is a D878A, H879A, or N902A mutation.

50. The HSV-based vector of claim 36, wherein the mutant Cas9 polypeptide sequence is from Neisseria meningitidis and comprises a mutation in the RuvC domain.

51. The HSV-based vector of claim 50, wherein the mutation is a D16A mutation.

52. The HSV-based vector of claim 36, wherein the mutant Cas9 polypeptide sequence is from Neisseria meningitidis and comprises a mutation in the HNH domain.

53. The HSV-based vector of claim 52, wherein the mutation is a D587A, H588A, or N611A mutation.

54. The HSV-based vector of claim 36, wherein the mutant Cas9 polypeptide sequence is from Staphylococcus aureus and comprises a mutation in the RuvC domain.

55. The HSV-based vector of claim 54, wherein the mutation is a D10A mutation.

56. The HSV-based vector of claim 36, wherein the mutant Cas9 polypeptide sequence is from Staphylococcus aureus and comprises a mutation in the HNH domain.

57. The HSV-based vector of claim 56, wherein the mutation is a N580A mutation.

58. The HSV-based vector of any one of claims 33 to 57, wherein the Cas9 is a human codon optimized Cas9.

59. The HSV-based vector of any one of claims 1 to 33, wherein the first expression cassette comprises a polynucleotide encoding a Cpf1 polypeptide.

60. The HSV-based vector of claim 59, wherein the first expression cassette comprises a polynucleotide encoding a Cpf1 polypeptide isolated from Francisella novicida, Acidaminococcus sp. BV3L6, or Lachnospiraceae bacterium ND2006.

61. The HSV-based vector of claim 59 or 60, wherein the Cpf1 polypeptide comprises one or more mutations in a RuvC-like endonuclease domain.

62. The HSV-based vector of claim 61, wherein the mutant Cpf1 polypeptide sequence is from Francisella novicida and comprises a mutation in the RuvC-like domain.

63. The HSV-based vector of claim 62, wherein the mutation is a D917A, E1006A, or D1225A mutation.

64. The HSV-based vector of claim 33, wherein the CRISPR-Cas endonuclease is a Cas9 fusion polypeptide or a Cpf1 fusion polypeptide.

65. The HSV-based vector of claim 64, wherein the fusion polypeptide comprises one or more functional domains.

66. The HSV-based vector of claim 65, wherein the one or more functional domains is selected from the group consisting of: a histone methylase or demethylase domains, a histone acetylase or deacetylase domains, a SUMOylation domain, an ubiquitylation or deubiquitylation domain, a DNA methylase or DNA demethylase domain, and a nuclease domain.

67. The HSV-based vector of claim 66, wherein the nuclease domain is a FOK I nuclease domain.

68. The HSV-based vector of claim 66, wherein the nuclease domain is a TREX2 nuclease domain.

69. The HSV-based vector of any of the preceding claims, wherein the switch polypeptide comprises a TREX2 domain or is a polypeptide comprising a self-cleaving viral peptide and TREX2.

70. The HSV-based vector of any one of preceding claims, wherein the one or more guide RNAs are single strand guide RNAS (sgRNAs).

71. The HSV-based vector of any one of preceding claims, wherein the one or more guide RNAs are crRNAs.

72. The HSV-based vector of claim 71, wherein the second expression cassette further comprises one or more tracRNAs.

73. The HSV-based vector of any one of preceding claims, wherein the second expression cassette further comprises one or more RNA polymerase III promoters operably linked to the one or more guide RNAs.

74. The HSV-based vector of claim 73, wherein the second expression cassette further comprises one RNA polymerase III promoter operably linked to the one or more guide RNAs.

75. The HSV-based vector of claim 73, wherein the second expression cassette further comprises one RNA polymerase III promoter operably linked to each of the one or more guide RNAs.

76. The HSV-based vector of claim 73, wherein the second expression cassette further comprises one RNA polymerase III promoter operably linked to each of the one or more guide RNAs.

77. The HSV-based vector of and one of claims 73-76, wherein the second expression cassette further comprises an RNA polymerase III promoter and at least one regulatory element for inducible expression operably linked to the one or more polynucleotides encoding the one or more guide RNAs.

78. The HSV-based vector of any one of claims 73-77, wherein the RNA polymerase III promoter is selected from the group consisting of: a human U6 snRNA promoter, a mouse U6 snRNA promoter, a human H1 RNA promoter, a mouse H1 RNA promoter, and a human tRNA-val promoter.

79. The HSV-based vector of claim 77 or claim 78, wherein the at least one regulatory element for inducible expression is selected from the group consisting of: a tetracycline responsive regulatory element and a peroxide inducible regulatory element.

80. The HSV-based vector of any one of preceding claims, wherein the third expression cassette comprises a polynucleotide encoding a switch polypeptide selected from the group consisting of: a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

81. The HSV-based vector of any one of preceding claims, wherein the third expression cassette comprises an RNA polymerase II promoter operably linked to the polynucleotide encoding the switch polypeptide.

82. The HSV-based vector of claim 81, wherein the RNA polymerase II promoter is a ubiquitous promoter.

83. The HSV-based vector of claim 82, wherein the ubiquitous promoter is selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-MN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

84. The HSV-based vector of claim 81, wherein the RNA polymerase II promoter is a tissue-specific or lineage-specific promoter.

85. The HSV-based vector of claim 84, wherein the tissue-specific or lineage-specific promoter is selected from the group consisting of: a B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), and a Wiskott-Aldrich syndrome protein (WASP) promoter (hematopoietic cell expression).

86. The HSV-based vector of claim 84, wherein the tissue-specific or lineage-specific promoter is selected from the group consisting of: an hSYN1 promoter, a TRPV1 promoter, a Nᵥ1.7 promoter, a Naᵥ1.8 promoter, and a Nᵥ1.9 promoter.

87. The HSV-based vector of claim 84, wherein the tissue-specific or lineage-specific promoter is a promoter specific for cell types found in the brain (e.g., neurons, glial cells), peripheral nervous system (e.g. dorsal root ganglia, trigeminal ganglia), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), or the like

88. The HSV-based vector of any one of the preceding claims, wherein the third expression cassette comprises at least one regulatory element for inducible expression that is operably linked to the polynucleotide encoding the switch polypeptide.

89. The HSV-based vector of claim 88, wherein at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

90. The HSV-based vector of any one of claims 4-89, further comprising a fourth expression cassette that comprises at least one regulatory element for inducible expression and a polynucleotide encoding a recombinase; wherein the polynucleotide encoding the CRISPR-Cas endonuclease comprises a transcription stop site flanked by one or more recombinase recognition sites.

91. The HSV-based vector of claim 90, wherein the one or more recombinase recognition sites selected from the group consisting of: LoxP, Lox511, Lox5171, Lox2272, m2, Lox71, Lox66, FRT, F₁, F₂, F₃, F₄, F₅, FRT(LE), FRT(RE), attB, attP, attL, and attR.

92. The HSV-based vector of claim 91, wherein the recombinase is selected from the group consisting of: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

93. The HSV-based vector of any one of claims 4-90, wherein the polynucleotide encoding the CRISPR-Cas endonuclease further encodes an inhibitory RNA and a binding site for the inhibitory RNA.

94. The HSV-based vector of claim 93, wherein the inhibitory RNA is a miRNA or a mishRNA.

95. The HSV-based vector of any one of the preceding claims, wherein the one or more guide RNAs are design to alter at least one site in a genome, optionally wherein the sequence of the one or more guide RNAs is selected from the group consisting of SEQ ID NOs: 1-55.

96. The HSV-based vector of any one of the preceding claims, further comprising a polynucleotide encoding a template for altering at least one site in a genome.

97. An HSV-based vector as shown in any one of the figures or embodiments disclosed herein.

98. An HSV-based vector of any one of the preceding claims for use editing one or more genomic sequences in a cell.

99. A cell comprising an HSV-based vector according to any one of claims 1-94.

100. A composition comprising an HSV-based vector according to any one of claims 1-98 or the cell of claim 99.

101. A method of genetically modifying a cell comprising introducing an HSV-based vector according to any one of claims 1-98 into the cell and inducing the expression of the switch polypeptide for a time sufficient to edit the genome of the cell.

102. The method of claim 101, wherein the editing of the genome in the cell comprises insertion of a regulatable transcriptional regulatory element upstream of a transcription start site in a gene of the cell.

103. The method of claim 102, wherein the transcriptional regulatory element is activated in the presence of an exogenous ligand or small molecule.

104. The method of claim 102, wherein the transcriptional regulatory element is activated in the absence of an exogenous ligand or small molecule.

105. The method of claim 102, wherein the transcriptional regulatory element is repressed in the presence of an exogenous ligand or small molecule.

106. The method of claim 102, wherein the transcriptional regulatory element is repressed in the absence of an exogenous ligand or small molecule.

107. The method of any one of claims 102 to 106 wherein the transcriptional regulatory element is inserted upstream of a gene associated with the regulation of pain.

108. The method of any one of claims 102 to 106, wherein the transcriptional regulatory element is inserted upstream of a gene encoding a voltage gated sodium channel.

109. The method of claim 108, wherein the voltage gated sodium channel is selected from the group consisting of: Naᵥ1.1, Naᵥ1.3, Naᵥ1.6, Naᵥ1.7, Naᵥ1.8, and Naᵥ1.9.

110. A method of using an HSV-based vector according to any one of claims 1-98, a cell according to claim 99, or a composition according to claim 90 to treat, prevent, or ameliorate at least one symptom of a monogenetic disease, to modify checkpoint genes in T cells used for T cell therapy, to modify nociceptive genes, to modify genes in viral genomes, to modify genes involved in neurodegenerative diseases, to modify genes involved in polycystic kidney disease or Walker-Warburg syndrome, to modify genes involved in trinucleotide repeat diseases, to modify genes involved in inflammatory disease, to modify genes involved in cancer, to modify genes involved in liver disease, to modify genes involved in retinal diseases, polynucleotide sequences that contribute to aberrant splicing, and to modify genes interregulatory genes.

111. A method of using an HSV-based vector according to any one of claims 1-98 to knockout or disrupt a gene or genetic regulatory sequence encoding a checkpoint gene in a T cell.

112. A method of using an HSV-based vector according to any one of claims 1-98 to knockout or disrupt a gene or genetic regulatory sequence, correct a sequence in the genome, or insert genetic material into the genome to treat a hemoglobinopathy in a subject.

113. A method of using an HSV-based vector according to any one of claims 1-98 to knockout or disrupt a nociceptive gene to treat neuropathic pain.

114. The method of claim 113, wherein the nociceptive gene encodes a voltage gated sodium channel.

115. The method of claim 114, wherein the voltage gated sodium channel is selected from the group consisting of: Naᵥ1.1, Naᵥ1.3, Naᵥ1.6, Naᵥ1.7, Naᵥ1.8, and Naᵥ1.9.

116. A method of using an HSV-based vector according to any one of claims 1-98 to knockout or disrupt a viral gene for the destruction of latent viral genomes, e.g., from HIV, HSV, or HPV.

117. A method of using an HSV-based vector according to any one of claims 1-98 to knockout or disrupt a region of trinucleotide repeat expansion in a gene involved in neurodegenerative diseases.

118. A method of using an HSV-based vector according to any one of claims 1-98 to knockout or disrupt a region involved in polycystic kidney disease or Walker-Warburg syndrome.

119. A method of using an HSV-based vector according to any one of claims 1-98 to knockout or disrupt CSF-1 production to deplete microglia and treat neuroinflammatory diseases such as Alzheimer's Disease and Parkinson's Disease.

120. A method of using an HSV-based vector according to any one of claims 1-98 to correct a sequence in FVIII or insert a copy of a normal copy of the FVIII gene in a cell in order to treat hemophilia.

121. A method of using an HSV-based vector according to any one of claims 1-98 to correct a sequence in the genome, or insert genetic material into the genome to treat a retinal disease, e.g, correct a rhodopsin mutation to treat retinitis pigmentosa or correct a mutation in ABCA4 to treat Stargardt disease.

122. A method of using an HSV-based vector according to any one of claims 1-98 to correct a sequence in the genome, or insert genetic material into the genome to treat a disease resulting from aberrant splicing.

123. A method of using an HSV-based vector according to any one of claims 1-98 to knockout or disrupt a gene or genetic regulatory sequence, correct a sequence in the genome, or insert genetic material into the genome to alter an interregulatory gene.

124. A method of using an HSV-based vector according to any one of claims 1-98 to treat Aicardi-Goutières Syndrome; Alexander Disease; Allan-Herndon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; or Xeroderma Pigmentosum.

125. A method of using an HSV-based vector according to any one of claims 1-98 to treat a monogenic disease, disorder, or condition.

126. The method of claim 125, wherein the monogenic disease, disorder, or condition is 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis; anauxetic dysplasia; androgen insentivity syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT1 deficiency; glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborm errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome Imag; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1 Imag; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann corneal dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCA5; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; or Zellweger disease.

127. A method of using an HSV-based vector according to any one of claims 1-98 to treat macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

128. The HSV-based vector according to any one of the preceding claims, wherein the HSV-based vector is an HSV-1 based vector or an HSV-2 based vector.
